(19) United States
(12) Patent Application Publication
(10) Pub. No.: US 2019/0117690 A1
(43) Pub. Date: Apr. 25, 2019

(54) USE OF HETERODIMERIC IL-15 IN ADOPTIVE CELL TRANSFER

(71) Applicant: THE UNIVERSITY STATE OF AMERICA AS REPRESENTED BY THE SECRETARY OF THE DEPARTMENT OF HEALTH AND HUM, Rockville, MD (US)

(72) Inventors: George N. Pavlakis, Rockville, MD (US); Barbara K. Felber, Rockville, MD (US); Cristina Bergamaschi, Urbana, MD (US)

(73) Assignee: THE UNIVERSITY STATE OF AMERICA AS REPRESENTED BY THE SECRETARY OF THE DEPARTMENT OF HEALTH AND HUM, Rockville, MD (US)

(21) Appl. No.: 16/091,967

(22) PCT Filed: Apr. 6, 2017
(86) PCT No.: PCT/US2017/026447

§ 371 Date: Oct. 5, 2018

Related U.S. Application Data
(60) Provisional application No. 62/319,259, filed on Apr. 6, 2016.
(60) Provisional application No. 62/497,948, filed on Dec. 8, 2016.

(51) Int. Cl. A61K 35/17; A61K 38/20; A61P 35/00; A61K 38/17; C12N 15/85; C12N 15/62; C07K 14/54; C07K 14/715; A61K 9/00

(52) U.S. Cl. CPC A61K 35/17; A61K 38/2086; A61P 35/00; A61K 38/1793; A61K 9/0019; C12N 15/62; C07K 14/5443; C07K 14/7155; C12N 15/85

(57) ABSTRACT

The disclosure provides methods of performing adoptive cell transfer using IL-15, where the methods are performed without lymphodepletion of the subject.

### CROSS-REFERENCE TO RELATED APPLICATIONS

This application claims benefit of priority of U.S. Provisional Application No. 62/319,259, filed Apr. 6, 2016 and U.S. Provisional Application No. 62/497,948, filed Dec. 8, 2016, each of which applications is herein incorporated by reference for all purposes.

### REFERENCE TO A SUBMISSION OF A SEQUENCE LISTING AS AN ASCII TEXT FILE

This application includes a Sequence Listing as a text file named “077867-1040787-628200PC_SequenceListing.txt” created on Apr. 5, 2017 and containing 12,330 bytes. The material contained in this text file is hereby incorporated by reference in its entirety for all purposes.

### BACKGROUND OF THE INVENTION

Adoptive immunotherapy with tumor-specific T cells either isolated from tumor tissue or engineered to recognize tumor-associated antigens is a promising approach for cancer immunotherapy (1-6). Studies in mice and humans have shown that the effectiveness of adoptive cell transfer (ACT) therapy can be improved by lymphodepleting pre-treatment of the host (7-10). Several mechanisms have been proposed for this beneficial effect. Previous studies showed that lymphodepletion removes the cellular sink for homeostatic cytokines and allows free cytokines to induce survival and proliferation of adoptively transferred cells (11). In line with these findings, increased plasma levels of Interleukin-7 (IL-7) and Interleukin-15 (IL-15) were measured in humans undergoing lymphodepleting regimens (10). The cytoreductive treatment also results in depletion of Tregs and myeloid derived suppressor cells (MDSC), associated with immune suppression and tolerance (3, 12). However, in humans, T cell recovery after lymphodepletion treatment may be delayed and incomplete (13-15), and may lead to severe and prolonged immune dysfunction and significant morbidity and mortality from opportunistic and recurrent infections (16, 17). Delays in immune reconstitution can also contribute to the relapse of malignant disease. Therefore, although lymphopenia creates a modified immune physiology that can favor the effectiveness of adoptive immunotherapy, the negative consequences of T cell depletion could offset the benefits.

ACT therapy benefits from the provision of exogenous γ chain cytokines that play an important role in promoting differentiation, proliferation and survival of the adoptively transferred T cells (18, 19). As a non-redundant member of this family of cytokines, IL-15 is important for the growth, mobilization and cytotoxicity of lymphocytes, including T and NK cells (20-23). Several studies have identified IL-15 as a key factor for the homeostatic proliferation of CD8+ T cells (24, 25) and evaluated its role in supporting ACT cell growth in vitro and in vivo. Klebanoff et al. demonstrated that pre-culturing with IL-15 resulted in the generation of anti-tumor CD8+ T cells with central memory phenotype. In comparison to IL-2, IL-15 is superior in inducing T clones with greater proliferative and cytokine secretion potential as well as effectiveness in inducing regression of established melanoma upon adoptive transfer in mice (26). IL-15 is also important for the in vivo persistence of the transferred cells. While ACT therapy resulted in tumor control in wild type mice, the effectiveness of the treatment was abrogated at about one month after cell transfer in IL-15 knock out (KO) mice, suggesting a role for endogenous IL-15 in promoting long-lasting efficacy of ACT therapy in a mouse model of melanoma (26). Similar results were also obtained in the macaque model, where CMV-specific CD8 autologous clones generated in the presence of IL-15 showed a central-memory phenotype rather than terminally differentiated effector phenotype as well as superior persistence (27). Additional findings also demonstrated a role of IL-15 in breaking tolerance and in rescuing tolerant T CD8 for use in adoptive immunotherapy of established tumors (28-30).

It has previously been shown that IL-15 is produced and functions as heterodimeric complex of two polypeptide chains, IL-15 and IL-15 Receptor alpha (IL-15Rα) (31). The two polypeptide chains are co-produced and form a complex in the endoplasmic reticulum, before they are fully glycosylated and traffic through the Golgi to the plasma membrane (32-34). Membrane-embedded IL-15Rα is responsible for IL-15 retention on the cell surface, where it is transpresented to adjacent responding cells expressing the IL-2/IL-15 receptor βγ (35). In addition, after a specific proteolytic cleavage of the IL-15Rα, a soluble heterodimeric form of IL-15 is released, circulates in the blood and is stable and biologically active (31, 33, 36). These data suggest that IL-15Rα is not a receptor for the IL-15 polypeptide chain, but the other half of heterodimeric IL-15 (hetIL-15) (37).

In view of adverse effects of lymphodepletion, there is a need for improved methods of adoptive cell transfer. This invention addresses this need.

### BRIEF SUMMARY OF THE INVENTION

The present invention provides methods of performing ACT comprising administering heterodimeric IL-15/IL-15Rα complexes. Not to be bound by theory, in many tumor types the number of CD8+ cells correlate with the outcome, indicating participation of the immune system in tumor clearance. HetIL-15 dramatically increases the number of lymphocytes in the tumor.

In one aspect, the disclosure relates to use of hetIL-15 in the absence of lymphodepletion to support adoptively transferred cells of any type. Further, hetIL-15 is superior to the lymphodepletion in that the sustained dosage of exogenous IL-15 increases the production of tumor antigen-specific cells and preferential infiltration into the tumor.

Therefore, the sustained administration of IL-15 provides an unexpected effect, which is the enrichment of tumor antigen-specific cells in the tumor.

HetIL-15 can be used in conjunction with any kind of adoptive cell transfer protocol. Thus, ACT may employ CD8+ T-lymphocytes, CD4+ T-lymphocytes, monocytes, dendritic cells, or Natural Killer cells or any combination of these and additional cell types. In some embodiments, the ACT cells are genetically modified, e.g., to express a native antigen receptor or a chimeric antigen receptor; or otherwise modified, e.g., to secrete cytokines or other anti-tumor molecules, to enhance anti-tumor activity of the ACT cells. In some embodiments, the cells used for ACT are derived from the subject receiving ACT.

In some aspects, the provided herein is a method of increasing adoptive cell therapy efficacy in a subject that does not undergo a lymphodepletion procedure, the method comprising: administering a heterodimeric IL-15/IL-15 receptor alpha complex (hetIL-15) to the subject; and administering adoptive cell transfer (ACT) cells to the subject, wherein hetIL-15 is administered at a frequency and in an amount that increases the number of lymphocytes present in the tumor. In some embodiments, hetIL-15 is administered for at least 10 days. In some embodiments, hetIL-15 is administered every day, or at two-day intervals or at three-day intervals. In some embodiments, hetIL-15 is administered at longer intervals, e.g., four-day, five-day, or six-day intervals. In some embodiments, hetIL-15 is administered weekly. In some embodiments, hetIL-15 is administered subcutaneously. In some embodiments, hetIL-15 is administered intravenously. In some embodiments, the ACT cells comprise CD8+ T cells. In some embodiments, the ACT cells comprise Natural Killer cells. In some embodiments, the ACT cells are genetically modified to enhance anti-tumor effects. In some embodiments, the ACT cells are lymphocytes are not pre-treated in vitro with IL-12. In some embodiments, the subject is a human. In some embodiments, the hetIL-15 comprises a soluble IL-15Ra that is not fused to an Fc region. In some embodiments, the het 11-15 comprises an IL-15Ra-Fc fusion polypeptide.

### BRIEF DESCRIPTION OF THE DRAWINGS

FIGS. 1A-1D provide data illustrating that hetIL-15 promotes tumor infiltration and persistence of adoptively transferred Pmel-1 and endogenous CD8+ T cells in the absence of lymphodepletion. 1A: Schematic of the ACT therapy in B16 melanoma-bearing mice. 5×10⁶ Pmel-1 cells were adoptively transferred comparing 3 treatment protocols: (i) cell transfer without lymphodepletion (ACT, grey squares), (ii) cell transfer in irradiated host (ACT+XRT, white circles) and (iii) cell transfer plus IP hetIL-15 administration (ACT+hetIL-15, black triangles). Mice were sacrificed at day 5, 7 and 12 for tumor and spleen analysis. 1B: The frequency of tumor-infiltrating Pmel-1 cells was determined by flow cytometry at the indicated time points after ACT for each treatment group. The number of Pmel-1 cells in each tumor was normalized per million of cells present in the tumor suspension. Bars represent mean±SEM. Data of two independent experiments were combined. Statistical significance was calculated using one-way ANOVA. The p-values were corrected for multiple comparisons by using Holm-Sidak test (* p<0.05, ** p<0.01). 1C: The proportion of Pmel-1 cells present in the tumor overtime was calculated as percentage of the mean value at day 5 after ACT for each treatment group. Mean values±SEM are shown. For each treatment group, r² and significant deviation from zero were calculated by linear regression. Comparisons of the different treatment groups were performed using Two-Way ANOVA. The p-values were corrected for multiple comparisons by using Holm-Sidak test (**, p<0.01; ns, non-significant). 1D: The frequency of endogenous CD8+ T cells infiltrating the tumor was determined by flow cytometry at the indicated time points after ACT for each treatment group. The number of endogenous CD8⁺ T cells in each tumor was normalized per million of cells present in the tumor suspension. Individual animal values and mean±SEM are shown. Data of two independent experiments were combined. Statistical significance was calculated by using One-Way ANOVA. The p-values were corrected for multiple comparisons using Holm-Sidak test (** p<0.01).

FIG. 2 provides data illustrating that tumor-infiltrating Pmel-1 cells and endogenous CD8+ T cells localized within the tumor upon hetIL-15 treatment. Tumor infiltrating lymphocytes (TILs) were identified by immunohistochemistry staining using antibodies specific for CD3+, CD4+, CD8+, and CD90.1+(staining Pmel-1 cells). The mean values of the Pmel-1 cell (top panel) and endogenous CD8+ T cell (bottom panel) counts from 9-15 tumor images are shown. Five to six tumors in each treatment group were analyzed. Statistical significance was calculated by using One-Way ANOVA. The p-values were corrected for multiple comparisons by using Holm-Sidak test (*, p<0.05; **, p<0.01). InForm software was used to enumerate each cell type.

FIGS. 3A-3D provide data illustrating that tumor-resident Pmel-1 cells are preferentially targeted by hetIL-15. 3A: Fold difference in Pmel-1 and endogenous CD8+ T cell counts in tumor and spleen for mice in the ACT+hetIL-15 (left panel, black) and in the ACT+XRT (right panel, white) groups normalized to ACT alone. Bars represent mean fold change (±SEM) compared to the mean level of the animals in the ACT group (set as 1). Data were combined from three independent experiments (day 12 after ACT). Statistical significance was calculated using One-Way ANOVA. The p-values were corrected for multiple comparisons by using Holm-Sidak test (**, p<0.01). 3B: The percentage of Pmel-1 cells (defined by the expression of CD90.1) within the CD8+ T cell population was assessed by flow cytometry in tumor (left panels), spleen (middle panels) and lung (right panel) at day 5 and 12. A representative mouse from the ACT+hetIL-15 group is shown. 3C: The ratio of Pmel-1 cells to endogenous CD8 T cells in tumor, spleen, and lung of mice that receive ACT+hetIL-15 treatment was determined. Values from individual animals (combining data from day 5 and day 12 after ACT) and mean±SEM are shown. Data were combined from two independent experiments. Statistical significance was calculated using One-Way ANOVA. The p-values were corrected for multiple comparisons by using Holm-Sidak test (*, p<0.05; **, p<0.01). 3D: Mice implanted with B16 melanoma cells and MC38 colon carcinoma cells on opposite flanks underwent ACT+hetIL-15 treatment. Fold increase in Pmel-1/CD8⁺ T cell ratio was calculated for B16 tumor and MC38 tumor in comparison to spleen (set as 1) for each mouse. Analysis was performed at day 9 after ACT. Statistical significance was calculated using One-Way ANOVA. The p-values were corrected for multiple comparisons by using Holm-Sidak test (**, p<0.01).

FIGS. 4A-4C provide data illustrating that hetIL-15 increases cytotoxic potential and IFN-γ production of adoptively transferred Pmel-1 cells in the tumor. 4A: The frequency of GzmB⁺Pmel-1 cells in the tumor (% of total Pmel-1 cells) was determined by intracellular staining followed by flow cytometry. A representative animal for each treatment group is shown. 4B: The frequency of GzmB⁺Pmel-1 cell in tumors is expressed as the percentage of total Pmel-1 cells (left panel) and number of GzmB⁺Pmel-1 cells normalized per million of cells present in the tumor suspension (right panel); mean values±SEM are shown for the three groups. Data collected from day 7 and day 12 after ACT were combined. Statistical significance was assessed using One-Way ANOVA. The p-values were corrected for multiple comparisons by using Holm-Sidak test (*, p<0.05; **, p<0.01). 4C: The frequency of IFN-γ producing Pmel-1 cells (left) and endogenous CD8⁺ T cells (middle panel) in tumor and of Pmel-1 cells in inguinal lymph nodes (right) was determined upon 6 hours (tumor) or 12 hours (lymph node) ex vivo cultures in medium only or in presence of the hgp10025-33 peptide. In C, the bars are in the order, from left to right of ACT, ACT+XRT, and ACT+hetIL-15. Analysis was performed at day 7 after ACT. ACT: n=3: ACT+XRT: n=5 and ACT+hetIL-15: n=5. Statistical significance was assessed using One-Way ANOVA. The p-values were corrected for multiple comparisons by using Holm-Sidak test (*, p<0.05; **, p<0.01).

FIGS. 5A-5B provide data illustrating that hetIL-15 treatment decreases PD-1 expression on tumor infiltrating Pmel-1 cells. 5A: Expression of the surface maker PD-1 in spleen (solid grey) and tumor (black line) from a representative untreated B16 melanoma-bearing mouse. The geometric mean fluorescent intensity (gMFI) of PD-1 in tumor versus spleen cells was determined for untreated B16 melanoma-bearing mice (n=11). Individual animal values and mean±SEM are shown. Data from two independent experiments were combined. Statistical significance was calculated using unpaired student's t-test (**, p<0.01). 5B: The gMFI of PD-1 on Pmel-1 cells in the tumor (left) and spleen (right) was determined from animals treated with ACT+XRT (white) or ACT+hetIIL-15 (black). Values from individual animals and mean±SEM are shown. Data collected from day 7 and day 12 after ACT from two independent experiments were combined. Statistical significance was calculated using unpaired student's t-test (**, p<0.01; *, p<0.05).

FIGS. 6A-6D provide data illustrating that hetIL-15 treatment alleviates exhaustion of transferred Pmel-1 cells in the tumor and increases tumor Pmel-1/Treg ratio. 6A: Percentage of Pmel-1 cells in tumor expressing the proliferation marker Ki67 for the mice in each of the three treatment groups at day 12 after ACT. Bars represent mean±SEM. Data from two independent experiments were combined. Statistical significance was assessed using One-Way ANOVA. The p-values were corrected for multiple comparisons using Holm-Sidak test (**, p<0.01). 6B: Pmel-1 cells infiltrating the tumor were analyzed for the expression of PD-1, Ki67, and GzmB by flow cytometry. The GzmB+ Pmel-1 cells (black dots) were overlayed on the total Pmel-1 cell population (grey contour). A representative animal from the ACT (left panel), ACT+XRT (middle panel) and ACT+hetIL-15 (right panel) treatment groups at day 12 after ACT is shown. 6C: The percentage of proliferating and cytotoxic Pmel-1 cells characterized by low expression of PD-1 (PD-1lowGzmB+Ki67+) was determined in the tumor at day 12 after ACT (left panel). The percentage of Pmel-1 cells with a phenotype consistent with exhaustion (PD-1highGzmB-Ki67-) was also determined in the tumor at day 12 after ACT (right panel). The values from individual animal and mean±SEM are shown. Data from two independent combined experiments. Statistical significance was assessed using One-Way ANOVA. The p-values were corrected for multiple comparisons using Holm-Sidak test (**, p<0.01). 6D: The frequency of tumor-infiltrating Treg cells was determined by flow cytometry at day 12 after ACT for each treatment group. The number of Treg cells in each tumor was normalized per million of cells present in the tumor suspension. Bars represent mean±SEM (left panel). The Pmel-1/Treg ratio was determined in tumor at day 12 after ACT for each treatment group. Bars represent mean±SEM. **, p<0.01 (right panel).

FIGS. 7A-7B provide data illustrating that hetIL-15 and ACT promote tumor control in the absence of lymphodepletion 7A: Mice were implanted with 5×10⁵ B16 cells SC at day −5. Mice were randomized in four treatment groups: PBS administration (dashed black, n=10), ACT alone (grey, n=7), hetIL-15 alone (dashed grey, n=7) and ACT+hetIL-15 (black, n=8). Splenic derived Pmel-1 cells (1×10⁶/mouse) were administered at day 0. Injections of hetIL-15 were performed 3 times per week for a total of 8 doses (3 μg/dose/mouse). Tumor measurements were performed every 2 to 3 days. Mean±SEM per each time points are shown. A representative experiment of three is shown. Statistical significance was calculated using Two-Way ANOVA. The p-values were corrected for multiple comparisons by using Holm-Sidak test (** p<0.01). 7B: Survival (%) of mice in the different treatment groups was followed up to day 28, when all PBS-treated mice (No treatment) were sacrificed due to the large tumor mass. Difference in survival between the different groups was determined by Mantel-Cox Log-rank test (* p<0.05).

FIG. 8 shows that IL-2 co-administration with ACT results in tumor accumulation and proliferation of Pmel-1 cells similar to hetIL-15, but significantly increases the frequency of tumor-associated Tregs. 8A: 5×10⁶ Pmel-1 cells were adoptively transferred comparing three treatment protocols: cell transfer without lymphodepletion (ACT, grey symbols), cell transfer plus IP hetIL-15 administration (ACT+hetIL-15, black symbols), and cell transfer plus IP IL-2 administration (9 μg/dose, white symbols). Mice were sacrificed at day 10 for tumor analysis. The frequency of tumor-infiltrating Pmel-1 cells was determined by flow cytometry for each treatment group. The number of Pmel-1 cells in each tumor was normalized per million of cells present in the tumor suspension. Bars represent mean±SEM. * p<0.05, ** p<0.01. 8B: Percentage of Pmel-1 cells in tumor expressing the proliferation marker Ki-67 for the mice in each of the three treatment groups at day 10 after ACT. Bars represent mean±SEM. ** p<0.01. 8C: The frequency of tumor-infiltrating Tregs was determined by flow cytometry at day 10 after ACT for each treatment group. The number of Tregs in each tumor was normalized per million of cells present in the tumor suspension. Bars represent mean±SEM (left panel). * p<0.05. 8D: The Pmel-1/Treg ratio was determined in tumor for each treatment group at day 10 after ACT. Bars represent mean±SEM. ** p<0.01. 8E: Mice were implanted with 5×10⁵ B16 cells SC at day −5. Three treatment groups were compared: No treatment (grey, n=10), ACT+hetIL-15 (black, n=10) and ACT+IL-2 (white, n=10). Splenic derived Pmel-1 cells (1×10⁶/mouse) were administered at day 0. IP injections of hetIL-15 and IL-2 were performed 3 times per week for a total of 8 doses (3 μg/dose/mouse). Tumor measurements were performed every 2 to 3 days. Mean±SEM for each time points are shown.

FIG. 9 provides data illustrating that endogenous IL-15 accounts for increased proliferation of transferred CD8+ T cells in the lymphodepleted host. Purified CFSE-labeled T cells (from C57BL/6 spleen; 2×10⁷/mouse) were adoptively transferred into C57BL/6 wild type or IL-15 KO mice. The histograms represent the CFSE profile of donor CD8+ T cells isolated from spleens of a representative mouse of the untreated (upper panels) and 1 day after irradiation (bottom panels) group, analyzed on day 7 after ACT.

FIG. 10 shows a gating strategy for the identification of adoptively transferred Pmel-1 cells and endogenous CD8+ T cells infiltrating the tumor. The first gate for the identification of tumor-infiltrating lymphocytes was drawn on the basis of FSC and SSC to exclude debris and macrophages/granulocytes. After elimination of doublets, dead cells were excluded by gating on Live/Dead Dye negative events. The expression of CD45 was used to identify tumor-infiltrating lymphocytes. Within this population, adoptively transferred Pmel-1 cells were identified as CD3+CD8+CD90.1+ (black gate) and endogenous CD8+ T cells were identified as CD3+CD8+CD90.1− (grey gate).

FIG. 11 provides illustrative data showing absolute counts of splenic Pmel-1 and CD8+ T cells are profoundly affected by hetIL-15 treatment. B16 melanoma-bearing mice were randomized into 3 treatment groups: ACT (grey square), ACT+XRT (white circles) and ACT+hetIL-15 (black triangles). Mice were killed at the indicated time points after ACT and spleens were collected for analysis. The total number of Pmel-1 cells (A) and endogenous CD8+ T cells (B) per spleen was determined by flow cytometry overtime. Values of individual animals and mean±SEM are shown. Data from two independent experiments were combined.

FIG. 12 provides data illustrating that hetIL-15 treatment decreases PD-1 expression on endogenous CD8+ T cells. The gMFI of PD-1 on endogenous CD8+ T cells in the tumor (left) and spleen (right) was determined from animals treated with ACT+XRT (white) or ACT+hetIIL-15 (black). Values from individual animals and mean±SEM are shown. Data collected from day 7 and day 12 after ACT from two independent experiments were combined. Statistical significance was calculated by using unpaired student's t-test. (** p<0.01).

### DETAILED DESCRIPTION OF THE INVENTION

### Terminology

As used herein, the terms “about” and “approximately.” when used to modify a numeric value or numeric range, indicate that the numeric value or range as well as reasonable deviations from the value or range, typically 10% or 20% above and 10% or 20% below the value or range, are within the intended meaning of the recited value or range.

As used herein, the term “peak level” and “peak concentration” refer to the highest levels of free IL-15 in a sample (e.g., a plasma sample) from a subject over a period of time.

In certain embodiments, the period of time is the entire period of time between the administration of one dose of IL-15/IL-15Ra complex and another dose of the complex. In some embodiments, the period of time is approximately 24 hours, approximately 48 hours or approximately 72 hours after the administration of one dose of IL-15/IL-15Ra complex and before the administration of another dose of the complex.

As used herein, the terms “trough level” and “trough concentration” refer to the lowest levels of free IL-15 in a sample (e.g., a plasma sample) from a subject over a period of time. In certain embodiments, the period of time is the entire period of time between the administration of one dose of IL-15/IL-15Ra complex and another dose of the complex. In some embodiments, the period of time is approximately 24 hours, approximately 48 hours or approximately 72 hours after the administration of one dose of IL-15/IL-15Ra complex and before the administration of another dose of the complex.

As used herein, the term “normal levels” in the context of the concentration of free IL-15 refers to the concentration of free IL-15 found in a sample obtained or derived from a healthy subject. Basal plasma levels of free IL-15 in healthy subjects are approximately 1 pg/ml in humans and approximately 8-15 pg/ml in monkeys (such as macaques). Normal levels depend on the exact method used for measurement and may vary because of this.

As used herein, the phase “an effective ratio of IL-15 to lymphocyte cell number” means that the amount of IL-15 available for lymphocytes keeps pace with the number of lymphocytes so that lymphocytes continue proliferating or survive. In a specific embodiment, a trough concentration of approximately 1 pg/ml to 5 pg/ml, approximately 1 pg/ml to 10 pg/ml, approximately 1 pg/ml to 15 pg/ml, approximately 1 pg/ml to 20 pg/ml, approximately 1 to 25 pg/ml, approximately 1 pg/ml to 30 pg/ml, approximately 1 pg/ml to 40 pg/ml, or approximately 1 pg/ml to 50 pg/ml of free IL-15 in a plasma sample from a subject is indicative of “an effective ratio of IL-15 to lymphocyte cell number.” In a specific embodiment, a trough concentration of below 50 pg/ml, below 45 pg/ml, below 40 pg/ml, below 35 pg/ml, below 30 pg/ml, below 25 pg/ml, below 20 pg/ml, below 15 pg/ml, below 10 pg/ml, below 5 pg/ml, or below 1 pg/ml of free IL-15 in a plasma sample from a subject is indicative of “an effective ratio of IL-15 to lymphocyte cell number.” In another specific embodiment, a trough concentration above 50 pg/ml, 55 pg/ml, 60 pg/ml, 65 pg/ml, 70 pg/ml, 75 pg/ml, 80 pg/ml, 85 pg/ml, 90 pg/ml, 95 pg/ml, or 100 pg/ml of free IL-15 in a plasma sample from a subject is indicative that the ratio of iL-15 to lymphocyte cell number is excessive. In another specific embodiment, a trough concentration 50 pg/ml to 75 pg/ml, 60 pg/ml to 75 pg/ml, 75 pg/ml to 85 pg/ml, 75 pg/ml to 100 pg/ml, 85 pg/ml to 100 pg/ml or 50 pg/ml to 100 pg/ml of free IL-15 in a plasma sample from a subject is indicative that the ratio of IL-15 to lymphocyte cell number is excessive. Any method known to one skilled in the art for measuring free IL-15 concentration in a sample from a subject may be used, such as, e.g., an immunoassay. In a specific embodiment, an ELISA is used to measure the free IL-15 concentration in a sample from a subject.

As used herein, the terms “native IL-15” and “native interleukin-15” in the context of proteins or polypeptides refer to any naturally occurring mammalian interleukin-15 amino acid sequences, including immature or precursor and mature forms. In the present invention, a native IL-15 is preferably a primate IL-15 sequence and is typically a human IL-15 sequence. Non-limiting examples of GeneBank Accession Nos. for the amino acid sequence of various species of native mammalian interleukin-15 include NP 000576 (human, immature form), CAA62616 (human, immature form), AAB60398 (macaca mulatta, immature form), AAI00964 (human, immature form), and AAHI8149 (human). In one embodiment, the amino acid sequence of the immature/precursor form of native human IL-15, which comprises the long signal peptide (underlined) and the mature human native IL-15 (italicized), is provided:

(SEQ ID NO: 1)
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS.

In some embodiments, native IL-15 is the immature or precursor form of a naturally occurring mammalian IL-15. In other embodiments, native IL-15 is the mature form of a naturally occurring mammalian IL-15. In a specific embodiment, native IL-15 is the precursor form of naturally occurring human IL-15. In another embodiment, native IL-15 is the mature form of naturally occurring human IL-15. In one embodiment, the native IL-15 protein/polypeptide is isolated or purified.

As used herein, the terms “native IL-15” and “native “interleukin-15” in the context of nucleic acids refer to any naturally occurring nucleic acid sequences encoding mammalian interleukin-15, including the immature or precursor and mature forms. Nonlimiting examples of Gene Bank Accession Nos. for the nucleotide sequence of various species of native mammalian IL-15 include NM_000585 (human). In one embodiment, the nucleotide sequence encoding the immature/precursor form of native human IL-15, which comprises the nucleotide sequence encoding the long signal peptide (underlined) and the nucleotidesequence encoding the mature human native IL-15 (italicized), is provided:

(SEQ ID NO: 2)
atgagaatttcgaaacca catttgagaa gtatttccat
ccagtgctac ttgtgtttac ttctaaacag tcattttcta
actgaagctg gcattcatgtcttcattttg ggctgtttca
gtgcagggct tcctaaaaca gaagccaact gggtgactgt
aataagtgat ttgaaaaaaattgaagatct tattcaatct
atgcatattg atgctacttt atatacggaa agtgatgttc
accccagttg caaagtaacagcaatgaagt gctttctctt
ggagttacaa gttatttcac ttgagtccgg agatgcaagt
attcatgata cagtagaaaa tctgatcatc ctagcaaaca
acagtttgtc ttctaatggg aatgtaacag aatctggatg
caaagaatgt gaggaactggaggaaaaaaa tattaaagaa
tttttgcaga gttttgtaca tattgtccaa atgttcatca
acacttcttg a.

In a specific embodiment, the nucleic acid is an isolated or purified nucleic acid. In some embodiments, nucleic acids encode the immature or precursor form of a naturally occurring mammalian IL-15. In other embodiments, nucleic acids encode the mature form of a naturally occurring mammalian IL-15. In a specific embodiment, nucleic acids encoding native IL-15 encode the precursor form of naturally occurring human IL-15. In another embodiment, nucleic acids encoding native IL-15 encode the mature form of naturally occurring human IL-15.

As used herein, the terms “IL-15 derivative” and “interleukin-15 derivative” in the context of proteins or polypeptides refer to: (a) a polypeptide that is at least 40%, 45%, 50%, 55%, 60%, 65%, typically at least 70%, 75%, 80%, 85%, 90%, 95%, 98% or 99% identical to a native mammalian IL-15 polypeptide; (b) a polypeptide encoded by a nucleic acid sequence that is at least 40%, 45%, 50%, 55%, 60%, 65%, 70%, 75%, 80%, 85%, 90%, 95%, 98% or 99% identical a nucleic acid sequence encoding a native mammalian IL-15 polypeptide; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to a native mammalian IL-15 polypeptide; (d) a polypeptide encoded by nucleic acids that can hybridize under high, moderate or typical stringency hybridization conditions to nucleic acids encoding a native mammalian IL-15 polypeptide; (e) a polypeptide encoded by a nucleic acid sequence that can hybridize under high, moderate or typical stringency hybridization conditions to a nucleic acid sequence encoding a fragment of a native mammalian IL-15 polypeptide of at least 20 contiguous amino acids, at least 30 contiguous amino acids, at least 40 contiguous amino acids, at least 50 contiguous amino acids, at least 100 contiguous amino acids, or at least 150 contiguous amino acids; and/or (f) a fragment of a native mammalian IL-15 polypeptide. IL-15 derivatives also include a polypeptide that comprises the amino acid sequence of a naturally occurring mature form of a mammalian IL-15 polypeptide and a heterologous signal peptide amino acid sequence. In a specific embodiment, an IL-15 derivative is a derivative of a native human IL-15 polypeptide. In another embodiment, an IL-15 derivative is a derivative of an immature or precursor form of naturally occurring human IL-15 polypeptide. In another embodiment, an IL-15 derivative is a derivative of a mature form of naturally occurring human IL-15 polypeptide. In another embodiment, an IL-15 derivative is the IL-15N72D described in, e.g., Zhu et al., 2009. J. Immunol. 183: 3598 or U.S. Pat. No. 8,163,879. In another embodiment, an IL-15 derivative is one of the IL-15 variants described in U.S. Pat. No. 8,163,879. In one embodiment, an IL-15 derivative is isolated or purified.

In a preferred embodiment, IL-15 derivatives retain at least 50%, 55%, 60%, 65%, 70%, 75%, 80%, 85%, 90%, 95%, 98% or 99% of the function of native mammalian IL-15 polypeptide to bind IL-15Ra polypeptide, as measured by assays well known in the art, e.g., ELISA. Biacore, co-immunoprecipitation. In another preferred embodiment, IL-15 derivatives retain at least 50%, 55%, 60%, 65%, 70% 75%, 80%, 85%, 90%, 95%, 98% or 99% of the function of native mammalian IL-15 polypeptide to induce IL-15-mediated signal transduction, as measured by assays well-known in the art. e.g., electromobility shift assays, western blots, phosphoprotein analysis, ELISAs and other immunoassays. In a specific embodiment, IL-15 derivatives bind to IL-15Ra and/or IL-15Rβγ as assessed by, e.g., ligand/receptor binding assays well-known in the art.

Percent identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wis.). In a further specific embodiment, percent identity is determined using the BLAST algorithm. Information regarding hybridization conditions (e.g., high, moderate, and typical stringency conditions) has been described, see, e.g., U.S. Patent Application Publication No. US 2005/0048549 (e.g., paragraphs 72-73).

As used herein, the terms “IL-15 derivative” and “interleukin-15 derivative” in the context of nucleic acids refer to: (a) a nucleic acid sequence that is at least 40%, 45%, 50%, 55%, 60%, 65%, 70%, 75%, 80%, 85%, 90%, 95%, 98% or 99% identical to the naturally occurring nucleic acid sequence encoding a mammalian IL-15 polypeptide; (b) a nucleic acid sequence encoding a polypeptide that is at least 40%, 45%, 50%, 55%, 60%, 65%, 70%, 75%, 80%, 85%, 90%, 95%, 98% or 99% identical the amino acid sequence of a native mammalian IL-15 polypeptide; (c) a nucleic acid sequence that contains 1, 2, 3, 4, 5, 6, 7, 8, 9, 10, 11, 12, 13, 14, 15, 16, 17, 18, 19, 20 or more nucleic acid base mutations (i.e., additions, deletions and/or substitutions) relative to the naturally occurring nucleic acid sequence encoding a mammalian IL-15 polypeptide; (d) a nucleic acid sequence that hybridizes under high, moderate or typical stringency hybridization conditions to a naturally occurring nucleic acid sequence encoding a mammalian IL-15 polypeptide; (e) a nucleic acid sequence that hybridizes under high, moderate or typical stringency hybridization conditions to a fragment of a naturally occurring nucleic acid sequence encoding a mammalian IL-15 polypeptide; and/or (f) a nucleic acid sequence encoding a fragment of a naturally occurring nucleic acid sequence encoding a mammalian IL-15 polypeptide. In a specific embodiment, an IL-15 derivative in the context of nucleic acids is a derivative of a naturally occurring nucleic acid sequence encoding a human IL-15 polypeptide. In another embodiment, an IL-15 derivative in the context of nucleic acids is a derivative of a naturally occurring nucleic acid sequence encoding an immature or precursor form of a human IL-15 polypeptide. In another embodiment, an IL-15 derivative in the context of nucleic acids is a derivative of a naturally occurring nucleic acid sequence encoding a mature form of a human IL-15 polypeptide. In another embodiment, an IL-15 derivative in the context of nucleic acids is the nucleic acid sequence encoding the IL-15N72D described in, e.g., Zhu et al., 2009, J. Immunol. 183: 3598 or U.S. Pat. No. 8,163,879. In another embodiment, an IL-15 derivative in the context of nucleic acids is the nucleic acid sequence encoding one of the IL-15 variants described in U.S. Pat. No. 8,163,879.

IL-15 derivative nucleic acid sequences include codon-optimized/RNA-optimized nucleic acid sequences that encode native mammalian IL-15 polypeptide, including mature and immature forms of iL-15 polypeptide. In other embodiments, IL-15 derivative nucleic acids include nucleic acids that encode mammalian IL-15 RNA transcripts containing mutations that eliminate potential splice sites and instability elements (e.g., A/T or A/U rich elements) without affecting the amino acid sequence to increase the stability of the mammalian IL-15 RNA transcripts.

In a preferred embodiment, IL-15 derivative nucleic acid sequences encode proteins or polypeptides that retain at least 50%, 55%, 60%, 65%, 70%, 75%, 80%, 85%, 90%, 95%, 98% or 99% of the function of a native mammalian IL-15 polypeptide to bind IL-15Ra, as measured by assays well known in the art, e.g., ELISA. Biacore, coimmunoprecipitation or gel electrophoresis. In another preferred embodiment, IL-15 derivative nucleic acid sequences encode proteins or polypeptides that retain at least 50%, 55%, 60%, 65%, 70%, 75%, 80%, 85%, 90%, 95%. 98% or 99% of the function of a native mammalian IL-15 polypeptide to induce IL-15-mediated signal transduction, as measured by assays well-known in the art. e.g., electromobility shift assays, ELISAs and other immunoassays. In a specific embodiment, IL-15 derivative nucleic acid sequences encode proteins or polypeptides that bind to IL-15Ra and/or IL-15Rβγ as assessed by, e.g., ligand/receptor assays well-known in the art.

As used herein, the terms “IL-15” and “interleukin-15” refer to a native IL-15, an IL-15 derivative, or a native IL-15 and an IL-15 derivative.

As used herein, the terms “native IL-15Ra” and “native interleukin-15 receptor alpha” in the context of proteins or polypeptides refer to any naturally occurring mammalian interleukin-15 receptor alpha (“IL-15Ra”) amino acid sequence, including immature or precursor and mature forms and naturally occurring isoforms. Non-limiting examples of GeneBank Accession Nos. for the amino acid sequence of various native mammalian IL-15Ra include NP 002180 (human), ABK41438 (Macaca mulatta), and CA141082 (human). In one embodiment, the amino acid sequence of the immature form of the native full length P: human IL-15Ra, which comprises the signal peptide (underlined) and the mature human native IL-15Ra (italicized), is provided:

(SEQ ID NO: 3)
MAPRRARGCR TLGLPALLLL LLLRPPATRG ITCPPPMSVE
HADIWVKSYSLYSRERYICN SGFKRKAGTS SLTECVLNKA
TNVAHWTTPS LKCIRDPALV HQRPAPPSTVTTAGVTPQPE
SLSPSGKEPA ASSPSSNNTA ATTAAIVPGS QLMPSKSPST
GTTEISSHESSHGTPSQTTA KNWELTASAS HQPPGVYPQG
HSDTTVAIST STVLLCGLSA VSLLACYLKS RQTPPLASVE
MEAMEALPVT WGTSSRDEDL ENCSHHL.

The amino acid sequence of the immature form of the native soluble human IL-15Ra, which comprises the signal peptide (underlined) and the mature human native soluble IL-15Ra (italicized), is provided:

(SEQ ID NO: 4)
MAPRRARGCR TLGLPALLLL LLLRPPATRG ITCPPPMSVE
HADIWVKSYS LYSRERYICN SGFKRKA GTS SLTECVLNKA
TNVAHWTTPS LKCIRDPALV HQRPAPPSTV TTAGVTPQPE
SLSPSGKEPA ASSPSSNNTA ATTAAIVPGS QLMPSKSPST
GTTEISSHES SHGTPSQTTA KNWELTASAS HQPPGVYTQG.

See below for further discussion regarding the immature and mature forms of human native soluble IL-15Ra. In some embodiments, native IL-15Ra is the immature form of a naturally occurring mammalian IL-15Ra polypeptide. In other embodiments, native IL-15Ra is the mature form of a naturally occurring mammalian IL-15Ra polypeptide. In certain embodiments, native IL-15Ra is the naturally occurring soluble form of mammalian IL-15Ra polypeptide. In other embodiments, native IL-15Ra is the full-length form of a naturally occurring mammalian IL-15Ra polypeptide. In a specific embodiment, native IL-15Ra is the immature form of a naturally occurring human IL-15Ra polypeptide. In another embodiment, native IL-15Ra is the mature form of a naturally occurring human IL-15Ra polypeptide. In certain embodiments, native IL-15Ra is the naturally occurring soluble form of human IL-15Ra polypeptide. In other embodiments, native IL-15Ra is the full-length form of a naturally occurring human IL-15Ra polypeptide. In one embodiment, a native IL-15Ra protein or polypeptide is isolated or purified.

As used herein, the terms “native IL-15Ra” and “native interleukin-15 receptor alpha” in the context of nucleic acids refer to any naturally occurring nucleic acid sequences encoding mammalian interleukin-15 receptor alpha, including the immature or precursor and mature forms. Non-limiting examples of GeneBank Accession Nos. for the nucleotide sequence of various species of native mammalian IL-15Ra include NM_002189 (human), and EF033114 (Macaca mulatta). In one embodiment, the nucleotide sequence encoding the immature form of native human IL-15Ra, which comprises the nucleotide sequence encoding the signal peptide (underlined) and the nucleotide sequence encoding the mature human IL-15Ra (italicized), is provided:

(SEQ ID NO: 5)
atggcccc gcggcgggcg cgcggctgcc ggaccctcgg
tctcccggcg ctgctactgc tgctgctgct ccggccgccg
gcgacgcggg gcatcacgtg ccctcccccc atgtccgtgg
aacacgcaga catctgggtc aagagctaca gcttgtactc
cagggagcgg tacatttgtaactctggttt caagcgtaaa
gccggcacgt ccagcctgac ggagtgcgtg ttgaacaagg
ccacgaatgt cgcccactgg acaaccccca gtctcaaatg
cattagagac cctgccctgg ttcaccaaag gccagcgcca
ccctccacag taacgacggc aggggtgacc ccacagccag
agagcctctc cccttctgga aaagagcccg cagcttcatc
tcccagctca aacaacacag cggccacaac agcagctatt
gtcccgggct cccagctgat gccttcaaaa tcaccttcca
caggaaccac agagataagc agtcatgagt cctcccacgg
caccccctct cagacaacag ccaagaactg ggaactcaca
gcatccgcct cccaccagcc gccaggtgtg tatccacagg
gccacagcga caccactgtg gctatctcca cgtccactgt
cctgctgtgt gggctgagcg ctgtgtctct cctggcatgc
tacctcaagt caaggcaaac tcccccgctg gccagcgttg
aaatggaagc catggaggct ctgccggtga cttgggggac
cagcagcaga gatgaagact tggaaaactg ctctcaccac
ctatga.

The nucleotide sequence encoding the immature form of native soluble human IL-15Ra protein or polypeptide, which comprises the nucleotide sequence encoding the signal peptide (underlined) and the nucleotide sequence encoding the mature human soluble native IL-5Ra (italicized), is provided:

(SEQ ID NO: 6)
atggcccc gcggcgggcg cgcggctgcc ggaccctcgg
tctcccggcg ctgctactgc tgctgctgct ccggccgccg
gcgacgcggg gcatcacgtg ccctcccccc atgtccgtgg
aacacgcaga catctgggtc aagagctaca gcttgtactc
cagggagcgg tacatttgta actctggttt caagcgtaaa
gccggcacgt ccagcctgac ggagtgcgtg ttgaacaagg
ccacgaatgt cgcccactgg acaaccccca gtctcaaatg
cattagagac cctgccctgg ttcaccaaag gccagcgcca
ccctccacag taacgacggc aggggtgacc ccacagccag
agagcctctc cccttctgga aaagagcccg cagcttcatc
tcccagctca aacaacacag cggccacaac agcagctatt
gtcccgggct cccagctgat gccttcaaaa tcaccttcca
caggaaccac agagataagc agtcatgagt cctcccacgg
caccccctct cagacaacag ccaagaactg ggaactcaca
gcatccgcct cccaccagcc gccaggtgtg tatccacct gc.

In a specific embodiment, the nucleic acid is an isolated or purified nucleic acid. In some embodiments, naturally occurring nucleic acids encode the immature form of a naturally occurring mammalian IL-15Ra polypeptide. In other embodiments, naturally occurring nucleic acids encode the mature form of a naturally occurring mammalian IL-15Ra polypeptide. In certain embodiments, naturally occurring nucleic acids encode the soluble form of a naturally occurring mammalian IL-15Ra polypeptide. In other embodiments, naturally occurring nucleic acids encode the full-length form of a naturally occurring mammalian IL-15Ra polypeptide. In a specific embodiment, naturally occurring nucleic acids encode the precursor form of naturally occurring human IL-15 polypeptide. In another embodiment, naturally occurring nucleic acids encode the mature of naturally occurring human IL-15 polypeptide. In certain embodiments, naturally occurring nucleic acids encode the soluble form of a naturally occurring human IL-15Ra polypeptide. In other embodiments, naturally occurring nucleic acids encode the full-length form of a naturally occurring human IL-15Ra polypeptide.

As used herein, the terms “IL-15Ra derivative” and “interleukin-15 receptor alpha derivative” in the context of a protein or polypeptide refer to: (a) a polypeptide that is at least 40%, 45%, 50%, 55%, 60%, 65%, typically at least 70%, 75%, 80%, 85%, 90%, 95%, 98% or 99% identical to a native mammalian IL-15 polypeptide; (b) a polypeptide encoded by a nucleic acid sequence that is at least 40%, 45%, 50%, 55%, 60%, 65%, 70%, 75%, 80%, 85%, 90%, 95%, 98%/0 or 99% identical a nucleic acid sequence encoding a native mammalian IL-15Ra polypeptide; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to a native mammalian IL-15Ra polypeptide; (d) a polypeptide encoded by a nucleic acid sequence that can hybridize under high, moderate or typical stringency hybridization conditions to a nucleic acid sequence encoding a native mammalian IL-15Ra polypeptide; (e) a polypeptide encoded by a nucleic acid sequence that can hybridize under high, moderate or typical stringency hybridization conditions to nucleic acid sequences encoding a fragment of a native mammalian IL-15 polypeptide of at least 20 contiguous amino acids, at least 30 contiguous amino acids, at least 40 contiguous amino acids, at least 50 contiguous amino acids, at least 100 contiguous amino acids, or at least 150 contiguous amino acids; (f) a fragment of a native mammalian IL-15Ra polypeptide; and/or (g) a specific IL-15Ra derivative described herein. IL-15Ra derivatives also include a polypeptide that comprises the amino acid sequence of a naturally occurring mature form of mammalian IL-15Ra polypeptide and a heterologous signal peptide amino acid sequence. In a specific embodiment, an IL-15Ra derivative is a derivative of a native human IL-15Ra polypeptide. In another embodiment, an IL-15Ra derivative is a derivative of an immature form of naturally occurring human IL-15 polypeptide. In another embodiment, an IL-15Ra derivative is a derivative of a mature form of naturally occurring human IL-15 polypeptide. In one embodiment, an IL-15Ra derivative is a soluble form of a native mammalian IL-15Ra polypeptide. In other words, in certain embodiments, an IL-15Ra derivative includes soluble forms of native mammalian IL-15Ra, wherein those soluble forms are not naturally occurring. An example of an amino acid sequence of a truncated, soluble form of an immature form of the native human IL-15Ra comprises the following signal peptide (underlined) and the following truncated form of human native IL-15Ra (italicized):

(SEQ ID NO: 7)
MAPRRARGCR TLGLPALLLL LLLRPPATRG ITCPPPMSVE
HADIWYKSYS LYSRERYICN SGFKRKAGTS SLTECVEVKA
TNVAHWTTPS LKCIRDPALV HQRPAPPSTV TTAGVTPQPE
SLSPSGKEPA ASSPSSNNTA ATTAAIVPGS QLMPSKSPST
GTTEISSHES SHGTPSQTTA KNWELTASAS HQPPGVYPQG HSDTT.

Other examples of IL-15Ra derivatives include the truncated, soluble forms of native human IL-15Ra described herein, or the sushi domain, which is the binding site to IL-15. In a specific embodiment, an IL-15Ra derivative is purified or isolated. In some embodiments a soluble IL-15 that is contained in hetIL-15 in accordance with the invention comprises the amino acid sequence of the exracellular domain of human IL-15Ra with one, two, three, four, five, six, seven, or eight amino acid substitutions and/or deletions in the amino acid sequence PQGHSDTT (SEQ ID NO:8) of human IL-15Ra such that cleavage by an endogenous protease that cleaves human IL-15Ra is inhibited. In some embodiments, a soluble form of human IL-Ra contained in hetIL-15 for use in the invention has as a C-terminal sequence (of the human IL-15Ra) PQGHSDTT (SEQ ID NO:8), PQGHSDT (SEQ ID NO:9), PQGHSD (SEQ ID NO: 10), PQGHS (SEQ ID NO:11), PQGH (SEQ ID NO: 12), or PQG.

In a preferred embodiment, IL-15Ra derivatives retain at least 50%, 55%, 60%, 65%, 70%, 75%, 80%, 85%, 90%, 95%, 98% or 99% of the function of a native mammalian IL-15Ra polypeptide to bind an IL-15 polypeptide, as measured by assays well known in the art, e.g., ELISA, Biacore, co-immunoprecipitation. In another preferred embodiment. IL-15Ra derivatives retain at least 50%, 55%, 60%, 65%, 70%, 75%, 80%, 85%, 90%/0, 95%, 98% or 99% of the function of a native mammalian IL-15Ra polypeptide to induce IL-15-mediated signal transduction, as measured by assays well-known in the art, e.g., electromobility shift assays, ELISAs and other immunoassays. In a specific embodiment, IL-15Ra derivatives bind to IL-15 as assessed by methods well-known in the art, such as, e.g., ELISAs.

As used herein, the terms “IL-15Ra derivative” and “interleukin-15 receptor alpha derivative” in the context of nucleic acids refer to: (a) a nucleic acid sequence that is at least 40%, 45%, 50%, 55%, 60%, 65%, 70%, 75%, 80%, 85%/0, 90%, 95%, 98%/0 or 99%/0 identical to the naturally occurring nucleic acid sequence encoding a mammalian IL-15Ra polypeptide; (b) a nucleic acid sequence encoding a polypeptide that is at least 40%, 45%, 50%, 55%, 600/%, 65%, 70%, 75%, 80%, 85%, 90%, 95%, 98% or 99% identical the amino acid sequence of a native mammalian IL-15Ra polypeptide; (c) a nucleic acid sequence that contains 1, 2, 3, 4, 5, 6, 7, 8, 9, 10, 11, 12, 13, 14, 15, 16, 17, 18, 19, 20 or more nucleic acid mutations (i.e., additions, deletions and/or substitutions) relative to the naturally occurring nucleic acid sequence encoding a mammalian IL-15Ra polypeptide; (d) a nucleic acid sequence that hybridizes under high, moderate or typical stringency hybridization conditions to a naturally occurring nucleic acid sequence encoding a mammalian IL-15Ra polypeptide; (e) a nucleic acid sequence that hybridizes under high, moderate or typical stringency hybridization conditions to a fragment of a naturally occurring nucleic acid sequence encoding a mammalian IL-15Ra polypeptide; (f) a nucleic acid sequence encoding a fragment of a naturally occurring nucleic acid sequence encoding a mammalian IL-15Ra polypeptide: and/or (g) a nucleic acid sequence encoding a specific IL-15Ra derivative described herein. In a specific embodiment, an IL-15Ra derivative in the context of nucleic acids is a derivative of a naturally occurring nucleic acid sequence encoding a human IL-15Ra polypeptide. In another embodiment, an IL-15Ra derivative in the context of nucleic acids is a derivative of a naturally occurring nucleic acid sequence encoding an immature form of a human IL-15Ra polypeptide. In another embodiment, an IL-15Ra derivative in the context of nucleic acids is a derivative of a naturally occurring nucleic acid sequence encoding a mature form of a human IL-15Ra polypeptide. In one embodiment, an IL-15Ra derivative in the context of nucleic acids refers to a nucleic acid sequence encoding a derivative of mammalian IL-15Ra polypeptide that is soluble. In certain embodiments, an IL-15Ra derivative in context of nucleic acids refers to a nucleic acid sequence encoding a soluble form of native mammalian IL-15Ra, wherein the soluble form is not naturally occurring. In some embodiments, an IL-15Ra derivative in the context of nucleic acids refers to a nucleic acid sequence encoding a derivative of human IL-15Ra, wherein the derivative of the human IL-15Ra is a soluble form of IL-15Ra that is not naturally occurring. An example of an IL-15Ra derivative nucleic acid sequence is the nucleotide sequence encoding the truncated, soluble, immature form of a native human IL-15Ra protein or polypeptide that comprises the following nucleotide sequence encoding the signal peptide (underlined) and the following nucleotide sequence encoding a truncated form of the mature human native IL-15Ra (italicized):

(SEQ ID NO: 13)
atggcccc gcggcgggcg cgcggctgcc ggaccctcgg
tctcccggcg ctgctactgc tgctgctgct ccggccgccg
gcgacgcggg gcatcacgtg ccctcccccc atgtccgtgg
aacacgcaga catctgggtc aagagctaca gcttgtactc
cagggagcgg tacatttgta actctggttt caagcgtaaa
gccggcacgt ccagcctgac ggagtgcgtg ttgaacaagg
ccacgaatgt cgcccactgg acaaccccca gtctcaaatg
cattagagac cctgccctgg ttcaccaaag gccagcgcca
ccctccacag taacgacggc aggggtgacc ccacagccag
agagcctctc cccttctgga aaagagcccg cagcttcatc
tcccagctca aacaacacag cggccacaac agcagctatt
gtcccgggct cccagctgat gccttcaaaa tcaccttcca
caggaaccac agagataagc agtcatgagt cctcccacgg
caccccctct cagacaacag ccaagaactg ggaactcaca
gcatccgcct cccaccagcc gccaggtgtg tatccacagg
gccacagcga caccact

In specific embodiments, an IL-15Ra derivative nucleic acid sequence is isolated or purified. IL-15Ra derivative nucleic acid sequences include RNA or codon-optimized nucleic acid sequences that encode native IL-15Ra polypeptide, including mature and immature forms of IL-15Ra polypeptide. In other embodiments, IL-15Ra derivative nucleic acids include nucleic acids that encode IL-15Ra RNA transcripts containing mutations that eliminate potential splice sites and instability elements (e.g., A/T or A/U rich elements) without affecting the amino acid sequence to increase the stability of the IL-15Ra RNA transcripts.

In a preferred embodiment. IL-15Ra derivative nucleic acid sequences encode proteins or polypeptides that retain at least 50%, 55%, 60%, 65%, 70%, 75%, 80%, 85%, 90%, 95%, 98% or 99% of the function of a native mammalian IL-15Ra polypeptide to bind IL-15, as measured by assays well known in the art, e.g., ELISA, Biacore, co-immunoprecipitation. In another preferred embodiment, IL-15Ra derivative nucleic acid sequences encode proteins or polypeptides that retain at least 50%, 55%, 60%, 65%, 70%, 75%, 80%, 85%, 90%, 95%, 98% or 99% of the function of a native mammalian IL-15Ra to induce IL-15-mediated signal transduction, as measured by assays well-known in the art, e.g., electromobility shift assays. ELISAs and other immunoassays. In a specific embodiment. IL-15Ra derivative nucleic acid sequences encode proteins or polypeptides that bind to IL-15 as assessed by methods well-known in the art, such as, e.g., ELISAs.

As used herein, the terms “IL-15Ra” and “interleukin-15 receptor alpha” refer to a native IL-15Ra, an IL-15Ra derivative, or a native IL-15Ra and an IL-15Ra derivative.

As used herein, the term “IL-15/IL-15Ra complex” refers to a complex comprising IL-15 and IL-15Ra covalently or noncovalently bound to each other. In a preferred embodiment, the IL-15Ra has a relatively high affinity for IL-15, e.g., a Kd of 10 to 50 pM as measured by a technique known in the art, e.g., KinEx A assay, plasma surface resonance (e.g., BIAcore assay). In another preferred embodiment, the IL-15/IL-15Ra complex induces IL-15-mediated signal transduction, as measured by assays well-known in the art, e.g., electromobility shift assays. ELISAs and other immunoassays. In some embodiments, the IL-15/IL-15Ra complex retains the ability to specifically bind to the βγ chain. In a specific embodiment, the IL-15/IL-15Ra complex is isolated from a cell. The term “hetIL-15” as used herein refers to a complex in which the IL-15Ra is a soluble form. In some embodiments, het IL-15 comprises a soluble form of IL-15Ra, such as a soluble IL-15Ra as described herein, e.g., at the preceding paragraph describing the terms an “IL-15Ra derivative” and “interleukin-15 receptor alpha derivative”, that is not fused to a soluble Fc region and thus is not an IL-15Ra-Fc fusion polypeptide. In some embodiments, het IL-15 comprises IL-15Ra in the form of an IL-15Ra-Fc fusion polypeptide.

As used herein, the terms “subject” and “patient” are used interchangeably and refer to a mammal, such as a non-primate (e.g., cows, pigs, horses, cats, dogs, rats etc.) and a primate (e.g., monkey and human), most preferably a human.

As used herein, the terms “purified” and “isolated” in the context of a compound or agent (including, e.g., proteinaceous agents) that is chemically synthesized refers to a compound or agent that is substantially free of chemical precursors or other chemicals when chemically synthesized. In a specific embodiment, the compound or agent is 60%, 65%, 75%, 80%, 85%, 90%, 95%, or 99% free (by dry weight) of other, different compounds or agents.

As used herein, the terms “purified” and “isolated” when used in the context of a compound or agent (including proteinaceous agents such as polypeptides) that can be obtained from a natural source, e.g., cells, refers to a compound or agent which is substantially free of contaminating materials from the natural source, e.g., cellular materials from the natural source, such as but not limited to cell debris, cell wall materials, membranes, organelles, the bulk of the nucleic acids, carbohydrates, proteins, and/or lipids present in cells. The phrase “substantially free of natural source materials” refers to preparations of a compound or agent that has been separated from the material (e.g., cellular components of the cells) from which it is isolated. Thus, a compound or agent that is isolated includes preparations of a compound or agent having less than about 30%, 20%, 10%, 5%, 2%, or 1% (by dry weight) of cellular materials and/or contaminating materials.

An “isolated” nucleic acid sequence or nucleotide sequence is one which is separated from other nucleic acid molecules which are present in a natural source of the nucleic acid sequence or nucleotide sequence. Moreover, an “isolated”, nucleic acid sequence or nucleotide sequence, such as a eDNA molecule, can be substantially free of other cellular material or culture medium when produced by recombinant techniques, or substantially free of chemical precursors when chemically synthesized. In certain embodiments, an “isolated” nucleic acid sequence or nucleotide sequence is a nucleic acid sequence or nucleotide sequence that is recombinantly expressed in a heterologous cell.

In some embodiments, the terms “nucleic acid”, “nucleotide” and “polynucleotide” refer to deoxyribonucleotides, deoxyribonucleic acids, ribonucleotides, and ribonucleic acids, and polymeric forms thereof, and include either single- or double-stranded forms. In certain embodiments, such terms include known analogues of natural nucleotides, for example, peptide nucleic acids (“PNA”s), that have similar binding properties as the reference nucleic acid. In some embodiments, such terms refer to deoxyribonucleic acids (e.g., eDNA or DNA). In other embodiments, such terms refer to ribonucleic acid (e.g., mRNA or RNA).

As used herein, the terms “protein(s)” and “polypeptide(s)” interchangeably to refer to a chain of amino acids linked together by peptide bonds. In some embodiments, the terms “protein(s)” and “polypeptide(s)” refer to a macromolecule which comprises amino acids that are linked together by peptide bonds.

### Introduction

In one aspect, the disclosure is based, in part, on the discovery that hetIL-15 can be administered in conjunction with ACT to induce lymphocytes in a tumor and to specifically enrich antigen-specific lymphocytes in a tumor. In further aspect, the disclosure relates, in part to the discovery that of exogenous IL-15 enhances ACT in the absence of lymphodepletion. Illustrative data as described herein demonstrated that administration of exogenous IL-15 in the form of an IL-15/IL-15Ra complex (hetIL-15) promoted infiltration and persistence of both adoptively transferred tumor antigen-reactive CD8+ T cells and endogenous CD8+ T cells into the tumor. Following irradiation, tumor antigen-reactive CD8+ T cells also localized to tumor sites efficiently, but their persistence was severely reduced in comparison to mice treated with hetIL-15. It was found that hetIL-15 treatment led to the preferential enrichment of tumor antigen-reactive CD8+ T cells in tumor sites in an antigen-dependent manner. hetIL-15 treatment also increased proliferation and the cytotoxic ability of tumor-infiltrating tumor antigen-reactive CD8+ T cells while reducing their PD-1 level, resulting in improved tumor control and survival benefit. Thus, hetIL-15 administration improved the outcome of ACT, including in a lymphodepleted host.

### Lymphodepletion

In the present disclosure, a heterodimeric IL-15/IL-15Ra complex is administered to a subject in conjunction with adoptive cell transfer, where the subject has not undergone a lymphodepletion regimen. Such protocols are clinically recognized protocols and include non-myeloablative lymphodepleting drug therapy prior to the transfer of adoptively transferred cells as well as irradiation. Illustrative non-myeloablative lymphodepletion protocols are described, e.g., in by Dudley, et al., J Clin Oncol 23:2346-2357, 2011). Other lymphodepleting protocols include whole-body irradiation.

### IL-15/IL-15Ra Complexes

IL-15/IL-15Ra complexes can be obtained using any methods, e.g., as described in U.S. Patent Application Publication No. 20150359853 and WO2016018920, each of which is incorporated by reference. Although the invention is illustrated using hetIL-15 in which the IL-15Ra is a soluble form of IL-15Ra, other forms IL-15/IL-15Ra complex may also be employed, e.g., embodiments in which the extracellular domain of IL-15Ra is fused to a soluble domain such as an Fc domain.

hetIL-15 may be formulated for administration by any method known to one of skill in the art, including but not limited to, parenteral (e.g., subcutaneous, intravenous, intraperitoneal, or intramuscular) and intratumoral administration. In one embodiment, the hetIL-15 is formulated for local or systemic parenteral administration. In a specific embodiment, hetIL-15 is formulated for subcutaneous or intravenous administration. In some embodiments, hetIL-15 can be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient (i.e., hetIL-15) may be in powder form for constitution with a suitable vehicle, e.g., sterile pyrogen-free water, before use.

HetIL-15 is administered to in an amount sufficient to induce lymphocyte migration into the tumor and to specifically enrich antigen-specific lymphocytes in the tumor. In one embodiment, hetIL-15 is administered in a dose of approximately 0.1 μg/kg to approximately 10 μg/kg or in a dose of approximately 0.1 μg/kg to approximately 50 μg/kg to a subject. In another embodiment, hetIL-15 is administered in a dose of approximately 0.1 μg/kg to approximately 10 μg/kg, approximately 0.1 μg/kg to approximately 20 μg/kg, approximately 10 μg/kg to approximately 20 μg/kg, approximately 20 μg/kg to approximately 40 μg/kg, or approximately 25 μg/kg to 50 μg/kg. In some embodiments, hetIL-15 is administered to a patient every day, e.g., at a dose of about approximately 0.1 μg/kg to approximately 20 μg/kg.

In some embodiments, hetIL-15 is administered every two days. In some embodiments, het IL-15 is administered every three days. In some embodiments, hetIL-15 is administered every four days, or every five day, or every six days, or every seven days, or every eight days, or every nine days, or every 10 days, or at longer intervals. In some embodiments, het IL-15 is administered every day, or every other day, or every three days, or every four days for at least 10 days or longer.

In some embodiments, an IL-15/IL-15Ra complex is administered. e.g., by parenteral injection, such as subcutaneous on intravenous injection, at recurring intervals for at least 10 days to a lymphoreplete subject undergoing ACT. In some embodiments, the complex is administered at recurring intervals for at least 11 days, at least 12 days, at least 13 days, at least 14 days, or at least 15 days. In some embodiments, the complex is administered at recurring intervals for at least 20 days or at least 21 days, at least 28 days, or longer. In some embodiments, het IL-15 is administered at a dose of approximately 0.1 μg/kg to approximately 10 μg/kg or in a dose of approximately 0.1 μg/kg to approximately 20 μg/kg to a subject. In another embodiment, hetIL-15 is administered in a dose of approximately 0.1 μg/kg to approximately 10 μg/kg, approximately 0.1 μg/kg to approximately 20 μg/kg, approximately 0.1 μg/kg to approximately 50 μg/kg, approximately 10 μg/kg to approximately 20 μg/kg, approximately 20 μg/kg to approximately 40 μg/kg, or approximately 25 μg/kg to 50 μg/kg. In some embodiments, the complex is administered daily. In some embodiments, the complex is administered at 2-day intervals. In some embodiments, the complex is administered at 3-day intervals. In some embodiments, the complex is administered every 4 days or every 5 days. In some embodiments, administration is every 6 days, or once a week. In some embodiments, administration is every 10 days or every 2 weeks. In some embodiments, het IL-15 is administered at a dosing as described herein for 2 weeks, 3 weeks, or 4 weeks intermittently, e.g., with a break of 1 week or 2 weeks between dosing period, or a break of 3 or 4 weeks between dosing periods.

In some embodiments, an IL-15/IL-15Ra complex is administered to a subject in a cyclical regimen, wherein each cycle of the cyclical regimen comprises: (a) administering a dose, e.g., by subcutaneous on intravenous administration, of the IL-15/IL-15Ra complex to the subject at a certain frequency for a first period of time, and (b) no administration of IL-15/IL-15Ra complex for a second period of time. In certain embodiments, the cyclical regimen is repeated 2, 3, 4, 5, 6, 7, 8, 9, 10 or more times. In some embodiments, the IL-15/IL-15Ra complex is administered at a frequency of every day, every other day, every 3, 4, 5, 6 or 7 days. In certain embodiments, the first and second periods of time are the same. In other embodiments, the first and second periods of time are different. In specific embodiments, the first period for administration of the IL-15/IL-15Ra complex is 1 week to 4 weeks long, 2 to 4 weeks, 2 to 3 weeks, or 1 to 2 weeks. In other embodiments, the first period for administration of the IL-15/IL-15Ra complex is 1 week, 2 weeks, 3 weeks or 4 weeks long. In some embodiments, the second period of time is 1 week to 2 months, 1 to 8 weeks, 2 to 8 weeks, 1 to 6 weeks, 2 to 6 weeks, 1 to 5 weeks, 2 to 5 weeks, 1 to 4 weeks, 2 to 4 weeks, 2 to 3 weeks, 1 to 2 weeks, 3 weeks, 2 weeks or 1 week long. In a specific embodiment, the dose of the first cycle and each subsequent cycle is 0.1 pg/kg to 1 pg/kg, 1 pg/kg to 5 pg/kg, or 5 pg/kg to 10 pg/kg. In another embodiment, the dose of the first cycle and each subsequent cycle is 0.1 pg/kg to 0.5 pg/kg, 1 pg/kg to 2 pg/kg, 1 pg/kg to 3 pg/kg, 2 pg/kg to 5 pg/kg, or 2 pg/kg to 4 pg/kg. In another embodiment, the dose of the first cycle and each subsequent cycle is 0.1 pg/kg, 0.25 pg/kg, 0.5 pg/kg, 1 pg/kg, 1.25 pg/kg, 1.5 pg/kg, 1.75 pg/kg, 2 pg/kg, 2.25 pg/kg, 2.5 pg/kg, 2.75 pg/kg, 3 pg/kg, 3.25 pg/kg, 3.5 pg/kg, 4 pg/kg, 4.25 pg/kg, 4.5 pg/kg, 4.75 pg/kg, or 5 pg/kg. In certain embodiments, the dose used during the first cycle of the cyclical regimen differs from a dose used during a subsequent cycle of the cyclical regimen. In some embodiments, the dose used within a cycle of the regimen varies. For example, the dose used within a cycle or in different cycles of the cyclical regimen may vary depending, e.g., upon the condition of the patient.

In one embodiment, hetIL-15 is administered to a subject in a cyclical regimen, wherein each cycle of the cyclical regimen comprises: (a) administering a dose of hetIL-15 to the subject a certain number of times per week for a first period of time; and (b) no administration of hetIL-15 for a second period of time. In certain embodiments, the dose of hetIL-15 administered during the first cycle of the cyclical regimen is sequentially escalated. For example, if hetIL-15 is administered to a subject 3 times per week for two weeks, then the dose administered to the subject the second time during the first cycle of the cyclical regimen is increased relative to the dose administered the first time, the dose administered to the subject the third time during the first cycle of the cyclical regimen is increased relative to the dose administered the second time, the dose administered to the subject the fourth time is increased relative to the dose administered the third time, the dose administered to the subject the fifth time is increased relative the dose administered the fourth time, and the dose administered to the subject the sixth time is increased relative to the dose administered the fifth time. In certain embodiments, the plasma levels of IL-15 and/or lymphocyte counts are monitored. In certain embodiments, the dose of hetIL-15 administered during the first cycle of the cyclical regimen is sequentially escalated if the subject does not have any side effects. In some embodiments, the dose of hetIL-15 administered during the first cycle of the cyclical regimen is sequentially escalated if the subject does not experience any adverse events. In some embodiments, hetIL-15 is administered 1, 2, 3, 4, 5, 6 or 7 days per week. In certain embodiments, the cyclical regimen is repeated 2, 3, 4, 5, 6, 7, 8, 9, 10 or more times. In some embodiments, the dose of hetIL-15 administered to the subject during the second cycle and/or other subsequent cycles remains the same as the last dose administered to the subject during the first cycle. In other embodiments, the dose administered to the subject during the second cycle and/or other subsequent cycles is increased or decreased relative to the last dose administered to the subject during the first cycle. In some embodiments, the first and second periods of time are the same. In other embodiments, the first and second periods of time are different. In specific embodiments, the first period for administration of the IL-15/IL-15Ra complex is 1 week to 4 weeks long, 2 to 4 weeks, 2 to 3 weeks, or 1 to 2 weeks. In other embodiments, the first period for administration of the IL-15/IL-15Ra complex is 1 week, 2 weeks, 3 weeks or 4 weeks long. In some embodiments, the second period of time is 1 week to 2 months, 1 to 8 weeks, 2 to 8 weeks, 1 to 6 weeks, 2 to 6 weeks, 1 to 5 weeks, 2 to 5 weeks, 1 to 4 weeks, 2 to 4 weeks, 2 to 3 weeks, 1 to 2 weeks, 3 weeks, 2 weeks or 1 week long.

In some embodiments, an IL-15/IL-15Ra complex is administered subcutaneously or intravenously, wherein each cycle of the cyclical regimen comprises: (a) administering a dose of the IL-15/IL-15Ra complex to the subject 3 times per week for a first period of time 2 weeks or more; and (b) no administration of IL-15/IL-15Ra complex for a second period of time, wherein the dose of the IL-15/IL-15Ra complex is sequentially increased each time the subject receives the complex during the first period. In certain embodiments, the dose of the IL-15/IL-15Ra administered the dose administered to the subject during the first cycle of the cyclical regimen is 0.1 μg/kg to 5 μg/kg, the dose administered to the subject the second time during the first cycle of the cyclical regimen is 5 μg/kg to 15 μg/kg, the dose administered to the subject the third time during the first cycle of the cyclical regimen is 15 μg/kg to 25 μg/kg, the dose administered to the subject the fourth time during the first cycle of the cyclical regimen is 25 μg/kg to 35 μg/kg, the dose administered to the subject the fifth time during the first cycle of the cyclical regimen is 35 μg/kg to 45 μg/kg, the dose administered to the subject the sixth time is 50 μg/kg or greater. In certain embodiments, the plasma levels of IL-15 and/or lymphocyte counts are monitored. In some embodiments, the subject is monitored for side effects such as a decrease in blood pressure and/or an increase in body temperature and/or an increase in cytokines in plasma. In certain embodiments, the dose of the IL-15/IL-15Ra complex administered during the first cycle of the cyclical regimen is sequentially escalated if the subject does not have any side effects. In some embodiments, the dose of the IL-15/IL-15Ra complex administered during the first cycle of the cyclical regimen is sequentially escalated if the subject does not experience any adverse events, such as grade 3 or 4 lymphopenia, grade 3 granulocytopenia, grade 3 leukocytosis (WBC>100,000/mm3), or organ dysfunction. In some embodiments, the IL-15/IL-15Ra is administered 1, 2, 3, 4, 5, 6 or 7 days per week. In certain embodiments, the cyclical regimen is repeated 2, 3, 4, 5, 6, 7, 8, 9, 10 or more times. In some embodiments, the dose of IL-15/IL-15Ra administered to the subject during the second cycle and/or other subsequent cycles remains the same as the last dose administered to the subject during the first cycle. In other embodiments, the dose administered to the subject during the second cycle and/or other subsequent cycle is increased or decreased relative to the last dose administered to the subject during the first cycle. In certain embodiments, the first and second periods of time are the same. In other embodiments, the first and second periods of time are different. In specific embodiments, the first period for administration of the IL-15/IL-15Ra complex is 1 week to 4 weeks long, 2 to 4 weeks, 2 to 3 weeks, or 1 to 2 weeks. In other embodiments, the first period for administration of the IL-15/IL-15Ra complex is 1 week, 2 weeks, 3 weeks or 4 weeks long. In some embodiments, the second period of time is 1 week to 2 months, 1 to 8 weeks, 2 to 8 weeks, 1 to 6 weeks, 2 to 6 weeks, 1 to 5 weeks, 2 to 5 weeks, 1 to 4 weeks, 2 to 4 weeks, 2 to 3 weeks, 1 to 2 weeks, 3 weeks, 2 weeks or 1 week long.

In another embodiment, provided herein is a method for treating or managing cancer in a human subject comprising: (a) administering subcutaneously or intravenously to the subject a dose of approximately 0.1 μg/kg, approximately 0.25 μg/kg, approximately 0.5 μg/kg, approximately 1 μg/kg, approximately 2 μg/kg, approximately 3 μg/kg, approximately 4 μg/kg, or approximately 5 μg/kg of an IL-15/IL-15Ra complex every 1, 2 or 3 days over a period of 1 week to 3 weeks; and (b) after a second period of 1 week to 2 months (or 8 weeks) in which no IL-15/IL-15Ra complex is administered to the subject, administering subcutaneously or intravenously to the subject a dose of approximately 0.1 μg/kg, approximately 0.25 μg/kg, approximately 0.5 μg/kg, approximately 1 μg/kg, approximately 2 μg/kg, approximately 3 μg/kg, approximately 4 μg/kg, or approximately 5 μg/kg of the IL-15/IL-15Ra complex every 1, 2 or 3 days over a third period of 1 week to 3 weeks. In a specific embodiment, the cancer is melanoma, renal cell carcinoma, lung cancer (e.g., non-small cell lung cancer) or colon cancer. In certain embodiments, the cancer is metastatic. In a specific embodiment, the cancer is metastatic melanoma, metastatic renal cell carcinoma, metastatic lung cancer (e.g., metastatic non-small cell lung cancer) or metastatic colon cancer.

In some embodiments, administer IL-15/IL-15Ra complex comprises administering at least one initial low dose of an IL-15/IL-15Ra complex to the subject; and administering successively higher doses of the IL-15/IL-15Ra complex to the subject, for example, if the concentration of free IL-15 in a sample (e.g., a plasma sample) obtained from the subject a certain period of time after the administration of a dose of the IL-15/IL-15Ra complex and before administration of another dose of the IL-15/IL-15Ra complex (e.g., approximately 24 hours to approximately 48 hours, approximately 24 hours to approximately 36 hours, approximately 24 hours to approximately 72 hours, approximately 48 hours to approximately 72 hours, approximately 36 hours to approximately 48 hours, or approximately 48 hours to 60 hours after the administration of a dose of the IL-15/IL-15Ra complex and before the administration of another dose of the IL-15/IL-15Ra complex) is within normal levels or less than normal levels. In a particular embodiment, the subject is a human subject. In some embodiments, hetIL-15 is administered to a human in a low dose of between 0.1 μg/kg and 1 μg/kg as determined based on the mass of single chain IL-15. In another embodiment, het IL-15 is administered in a low dose of between 0.1 μg/kg and 0.5 μg/kg as determined based on the mass of single chain IL-15. In another embodiment, hetIL-15 is administered in a low dose of about 0.1 μg/kg, 0.2 μg/kg, 0.3 μg/kg, 0.4 μg/kg, 0.5 μg/kg, 0.6 μg/kg, 0.7 μg/kg, 0.8 μg/kg, 0.9 μg/kg or 1 μg/kg as determined based on the mass of single chain IL-15. In certain embodiments, an initial low dose is administered 1, 2, 3, 4, 5, 6, 7, 8, 9, 10 or more times, or 1 to 3, 1 to 4, 1 to 5, 2 to 4, 2 to 5, 1 to 6, 2 to 6, 3 to 6, 4 to 6, 6 to 8, 5 to 8, or 5 to 10 times. In some embodiments, an initial low dose is administered 1, 2, 3, 4, 5, 6, 7, 8, 9, 10 or more times, or 1 to 3, 1 to 4, 1 to 5, 1 to 6, 2 to 4, 2 to 5, 2 to 6, 3 to 6, 4 to 6 or 6 to 8 times over a 5 to 7 day, 5 to 10 day, 7 to 12 day, 7 to 14 day, 7 to 21 day or 14 to 21 day period of time. In certain embodiments, successively higher doses are administered, e.g., successively higher doses of 1.2, 1.25, 1.3, 1.35, 1.4, 1.45, 1.5, 2, 2.5, 3, 3.5, 4, 4.5, 5, 5.5, or 6 times higher than the previous dose, or 1.2 to 2, 2 to 3, 2 to 4, 1 to 5, 2 to 6, 3 to 4, 3 to 6, or 4 to 6 times higher than the previous dose. In some embodiments, each successively higher dose is 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% higher than the previous dose. In some embodiments, each dose is administered 1, 2, 3, 4, 5, 6, 7, 8, 9, 10 or more times, or 1 to 3, 1 to 4, 1 to 5, 2 to 4, 2 to 5, 1 to 6, 2 to 6, 3 to 6, 4 to 6, 6 to 8, 5 to 8, or 5 to 10 times. In specific embodiments, each dose is administered at least 1, 2, 3, 4, 5, 6 or more times, or 1 to 3, 1 to 4, 1 to 5, 2 to 4, 2 to 5, 1 to 6, 2 to 6, 3 to 6, 4 to 6, 6 to 8, 5 to 8, or 5 to 10 times over a 5 to 7 day, 5 to 10 day, 7 to 12 day, 7 to 14 day, 7 to 21 day or 14 to 21 day period of time. In another specific embodiment, each dose is administered at least once and the subject is administered a dose three times per 7 day week (e.g., Monday, Wednesday and Friday).

In some embodiments, the method further comprises administering a maintenance dose of the IL-15/IL-15Ra complex to the subject, wherein the maintenance dose reaches trough levels of free IL-15 concentration of approximately 1 pg/ml to approximately 5 pg/ml, approximately 2 pg/ml to approximately 5 pg/ml, approximately 2 pg/ml to approximately 10 pg/ml, approximately 5 pg/ml to approximately 10 pg/ml, approximately 10 pg/ml to approximately 15 pg/ml, approximately 10 pg/ml to approximately 20 pg/ml, approximately 20 pg/ml to approximately 30 pg/ml, approximately 30 pg/ml to approximately 40 pg/ml, or approximately 40 pg/ml to approximately 50 pg/ml, approximately 1 pg/ml to 50 pg/ml or approximately 5 pg/ml to approximately 50 pg/ml in a blood sample from the subject. In a specific embodiment, the maintenance dose is equal to or less than the highest dose received by the subject during the dose escalation phase of the therapeutic regimen which does not result in one, two, or more, adverse events.

An Il-15/IL-15Ra complex may be administered to a subject in a pharmaceutical composition. In certain embodiments, the complex is the sole/single agent administered to the subject, other than the cells that are administered for ACT. In other embodiments, hetIL-15 is administered in combination with one or more other therapies (e.g., an antibody that immunospecifically binds to Her2 or another cancer antigen; or a checkpoint inhibitor, such as an antibody that binds to PD-1 or a ligand of PD-1 (e.g., PD-L1); or a checkpoint inhibitor that inhibits a checkpoint protein such as CTLA-4, PDL2, B7-H3, B7-H4, BTLA, HVEM, TIM3, GAL9, LAG3, VISTA, KIR, 2B4, CD160, CGEN-15049, CHK 1, CHK2, A2aR, or B-7 family ligands or a combination thereof.

### ACT

In its broadest sense, Adoptive cell therapy (ACT) is a treatment method where cells are removed from a donor, cultured and/or manipulated in vitro, and administered to a patient for the treatment of a disease. In some embodiments, cells administered in adoptive cell transfer are CD8+ T cells. Other cells that can be administered in ACT include CD4+ T-lymphocyte, monocyte(s), dendritic cell(s), or Natural Killer cell(s). In some embodiments, the cells used for ACT are derived from the subject receiving ACT.

### T Lymphocytes

T lymphocytes can be collected in accordance and enriched or depleted using techniques such as immunological-based selection methods using antibodies to desired surface antigens, e.g., flow cytometry and/or immunomagnetic selection. After enrichment and/or depletion steps, in vitro expansion of the desired T lymphocytes can be carried out. For example, the desired T cell population or subpopulation may be expanded by adding an initial T lymphocyte population to a culture medium in vitro, and then adding to the culture medium feeder cells, such as non-dividing peripheral blood mononuclear cells (PBMC), and incubating the culture (e.g., for a time sufficient to expand the numbers of T cells).

In some embodiments, T cells employed for ACT are not pretreated with IL-12.

The T lymphocytes collected and/or expanded include cytotoxic T lymphocytes (CTL), but may also include helper T lymphocytes that are specific for an antigen present on a human tumor or a pathogen. CD8+ cells can be obtained by using standard methods. In some embodiments, CD8+ cells are further sorted into naive, central memory, and effector cells by identifying cell surface antigens that are associated with each of those types of CD8+ cells.

Whether a cell or cell population is positive for a particular cell surface marker can be determined by flow cytometry using staining with a specific antibody for the surface marker and an isotype matched control antibody. A cell population negative for a marker refers to the absence of significant staining of the cell population with the specific antibody above the isotype control, positive refers to uniform staining of the cell population above the isotype control. In some embodiments, a decrease in expression of one or markers refers to loss of 1 log 10 in the mean fluorescence intensity and/or decrease of percentage of cells that exhibit the marker of at least 20% of the cells, 25% of the cells, 30% of the cells, 35% of the cells, 40% of the cells, 45% of the cells, 50% of the cells, 55% of the cells, 60% of the cells, 65% of the cells, 70% of the cells, 75% of the cells, 80% of the cells, 85% of the cells, 90% of the cell, 95% of the cells, and 100% of the cells and any % between 20 and 100% when compared to a reference cell population. In some embodiments, a cell population positive for a marker refers to a percentage of cells that exhibit the marker of at least 50% of the cells, 55° % of the cells, 60% of the cells, 65% of the cells, 70% of the cells, 75% of the cells, 80% of the cells, 85% of the cells, 90% of the cell, 95% of the cells, and 100% of the cells and any % between 50 and 100% when compared to a reference cell population.

CD4+ T helper cells are sorted into naive, central memory, and effector cells by identifying cell populations that have cell surface antigens. CD4+ lymphocytes can be obtained by standard methods.

Populations of CD4+ and CD8+ that are antigen-specific can be obtained by stimulating naive or antigen specific T lymphocytes with antigen. For example, antigen specific T cell clones can be generated to Cytomegalovirus antigens by isolating T cells from infected subjects and stimulating the cells in vitro with the same antigen. Naive T cells may also be used. Any number of antigens from tumor cells or cancer cells, or infectious agents may be utilized. Examples of such antigens include HIV antigens, HCV antigens, HBV antigens, CMV antigens, parasitic antigens, and tumor antigens such as orphan tyrosine kinase receptor ROR1, tEGFR, Her2, LI-CAM, CD 19, CD20, CD22, mesothelin, and CEA. In some embodiments, the adoptive cellular immunotherapy compositions are useful in the treatment of a disease or disorder including a solid tumor, hematologic malignancy, melanoma, or infection with a virus.

### Modification of T Lymphocyte Populations

In some embodiments it may be desired to introduce functional genes into the T cells to be used in immunotherapy in accordance with the present disclosure. For example, the introduced gene or genes may improve the efficacy of therapy by promoting the viability and/or function of transferred T cells; or they may provide a genetic marker to permit selection and/or evaluation of in vivo survival or migration; or they may incorporate functions that improve the safety of immunotherapy.

In embodiments, T cells are modified with chimeric antigen receptors (CAR). In some embodiments, CARs comprise a single-chain antibody fragment (scFv) that is derived from the variable heavy (VH) and variable light (VL) chains of a monoclonal antibody (mAb) linked to the TCR CD3+ chain that mediates T-cell activation and cytotoxicity.

Costimulatory signals can also be provided through the CAR by fusing the costimulatory domain of CD28 or 4-1 BB to the CD3+ chain. CARs are specific for cell surface molecules independent from HLA, thus overcoming the limitations of TCR-recognition including HLA-restriction and low levels of HLA-expression on tumor cells.

CARs can be constructed with specificity for any cell surface marker by utilizing antigen binding fragments or antibody variable domains of, for example, antibody molecules.

The antigen binding molecules can be linked to one or more cell signaling modules. In embodiments, cell signaling modules include CD3 transmembrane domain, CD3 intracellular signaling domains, and CD 28 transmembrane domains. In embodiments, the intracellular signaling domain comprises a CD28 transmembrane and signaling domain linked to a CD3 intracellular domain. In some embodiments, a CAR can also include a transduction marker such as EGFR

In embodiments, the intracellular signaling domain of the CD8+ cytotoxic T cells is the same as the intracellular signaling domain of the CD4+ helper T cells. In other embodiments, the intracellular signaling domain of the CD8+ cytotoxic T cells is different than the intracellular signaling domain of the CD4+ helper T cells.

In some embodiments, the CD8+ T cell and the CD4+ T cell are both genetically modified with an antibody heavy chain domain that specifically binds a pathogen-specific cell surface antigen. In embodiments, CARs are specific for cell surface expressed antigens associated with pathogens, tumors, or cancer cells. In some embodiments, a CAR is specific for an infectious disease antigen such as HIV, HCV, or HBV. In some embodiments, a CAR is specific for a tumor antigen such as orphan tyrosine kinase receptor ROR1, tEGFR, Her2, L1-CAM, CD19, CD20, CD22, mesothelin, and CEA. Methods for producing a CAR can be found, e.g., U.S. Pat. No. 6,410,319 by Forman and WO 2002/077029, U.S. Pat. No. 7,446,191, US2010/065818, US2010/025177, US2007/059298, and U.S. Pat. No. 7,514,537 by Jensen et al. and as described by Berger C. et al., J. Clinical Investigation, 118:1 294-308 (2008), which are hereby incorporated by reference.

In some embodiments, T cells can be modified with a recombinant T cell receptor. TCR could be specific for any antigen, pathogen or tumor. There are TCRs for many tumor antigens in melanoma (MARTI, gp100 for example), leukemia (WT1, minor histocompatibility antigens for example), breast cancer (her2, NY-BR1 for example). Various infection techniques have been developed which utilize recombinant infectious virus particles for gene delivery. This represents a currently preferred approach to the transduction of T lymphocytes of the present invention. The viral vectors which have been used in this way include virus vectors derived from simian virus 40, adenoviruses, adeno-associated virus (AAV), lentiviral vectors, and retroviruses. Thus, gene transfer and expression methods are numerous but essentially function to introduce and express genetic material in mammalian cells. Several of the above techniques have been used to transduce hematopoietic or lymphoid cells, including calcium phosphate transfection, protoplast fusion, electroporation, and infection with recombinant adenovirus, adeno-associated virus and retrovirus vectors. Primary T lymphocytes have been successfully transduced by electroporation and by retroviral infection.

Any suitable number of cells, e.g., T cells, such as CD8+ T cells, for ACT can be administered to a mammal, e.g. a human. In some embodiments at least 10⁴ or more, 10⁵ or more, 10⁶ or more, 10⁷ or more, 10⁸ or more, 10⁹ or more, or 10¹⁰ or more ⁺ T cells are administered.

A dose of the cells used in adoptive cell transfer can be administered to a mammal, e.g., a human, at one time or in a series of subdoses administered over a suitable period of time, e.g., on a daily, semi-weekly, weekly, bi-weekly, semi-monthly, bi-monthly, semi-annual, or annual basis, as needed. A dosage unit comprising an effective amount of a CD8⁺ T cell of the invention may be administered in a single daily dose, or the total daily dosage may be administered in two, three, four, or more divided doses administered daily, as needed.

With respect to an upper limit on the number of T cells that can be administered or the number of times that the T cells of the invention can be administered, one of ordinary skill in the art will understand that excessive quantities of administered T lymphocytes can lead to undesirable side effects and unnecessarily increase costs.

Cells for ACT, e.g., CD8+ T cells, administered in accordance with the invention may modified to express other polypeptides, such as chimerica antigen receptors and the like. In some embodiments, a preparation comprising cells for ACT does not substantially contain any other living cells

Anti-tumor activity of ACT cells, e.g., CD8+ T cells can be assessed in the presence or absence of hetIL-15. Illustrative protocols for determining activity are provided in the examples section. The effects of hetIL-15 on tumor infiltration by lymphocytes can be determined as described in the examples, for example, the numbers of tumor-infiltrating lymphocytes can be determined using immunohistochemistry, flow cytometry, or other methods.

Tumor growth and disease progression in a subject that is administered hetIL-15 in conjunction with ACT may be monitored during and after treatment of cancer via the subject methods of the present invention. Clinical efficacy can be measured by any method known in the art. In some embodiments, clinical efficacy of the subject treatment method is determined by measuring the clinical benefit rate (CBR). In some embodiments, the clinical benefit rate is measured by determining the sum of the percentage of patients who are in complete remission (CR), the number of patients who are in partial remission (PR) and the number of patients having stable disease (SD) at a time point at least 6 months out from the end of therapy. In some embodiments, CBR for the subject treatment method is at least about 50%. In some embodiments, CBR for the subject treatment method is at least about 55%, 60%, 65%, 70%, 75%, 80%, 85%, 90%, 95% or more.

Cells, e.g., T cells, for adoptive cell transfer and hetIL-15 may also be administered with other therapeutic agents, e.g., a chemotherapeutic agent or a biological agent.

Examples of chemotherapeutic agents which can be used in the compositions and methods of the invention include platinum compounds (e.g., cisplatin, carboplatin, and oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, and bendamustine), antitumor antibiotics (e.g., daunorubicin, doxorubicin, idarubicin, epirubicin, mitoxantrone, bleomycin, mytomycin C, plicamycin, and dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, and sunitinib), nitrosoureas (e.g., carmustine, fotemustine, and lomustine), hexamethylmelamine, mitotane, angiogenesis inhibitors (e.g., thalidomide and lenalidomide), steroids (e.g., prednisone, dexamethasone, and prednisolone), hormonal agents (e.g., tamoxifen, raloxifene, leuprolide, bicaluatmide, granisetron, and flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, and oxaprozin), selective cyclooxygenase-2 (COX-2) inhibitors, or any combination thereof.

Examples of biological agents that can be used in the compositions and methods of the invention include monoclonal antibodies (e.g., rituximab, cetuximab, panetumumab, tositumomab, trastuzumab, alemtuzumab, gemtuzumab ozogamicin, and bevacizumab), enzymes (e.g., L-asparaginase), growth factors (e.g., colony stimulating factors and erythropoietin), cancer vaccines, gene therapy vectors, or any combination thereof.

Combination therapy performed with ACT includes concurrent and successive administration of hetIL-15 and ACT. As used herein, hetIL-15 and ACT are said to be administered concurrently if they are administered to the patient on the same day, for example, simultaneously, or 1, 2, 3, 4, 5, 6, 7, or 8 hours apart, whereas hetIL-15 and ACT are said to be administered successively if they are administered to the patient on the different days, for example, administered at a 1-day, 2-day or 3-day intervals. In the methods described herein, administration of the IL-15/IL-15Ra complex can precede or follow ACT. In some embodiments, administration of hetIL-15 occurs before administration of ACT cells, e.g., at least 1 day before administration of ACT cells, or at least 2, at least 3, at least 4, at least 5, at least 6, or at least 7 days before administration of ACT cells. In some embodiments, administration of hetIL-15 occurs after administration of ACT cells, e.g., at least 1 day after administration of ACT cells, or at least 2, at least 3, at least 4, at least 5, at least 6, or at least 7 days after administration of ACT cells.

Cancers and related disorders that can be prevented, treated, or managed in accordance with the methods described herein include, but are not limited to, the following: Leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, and chronic lymphocytic leukemia, hairy cell leukemia; polycythemia Vera; lymphomas such as but not limited to Hodgkin's disease, and non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease, bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, and synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma: breast cancer including, but not limited to, adenocarcinoma, lobular (small cell) carcinoma intraductal carcinoma, medullary breast cancer, mucinous breast cancer, tubular breast cancer, papillary breast cancer, Paget's disease, and inflammatory breast cancer; adrenal cancer, including but not limited to, pheochromocytom and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, and acromegaly; eye cancers including but not limited to, ocular melanoma such as iris melanoma, choroidal melanoma, and cilliary body melanoma, and retinoblastoma; vaginal cancers, including but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease, cervical cancers including but not limited to, squamous cell carcinoma, and adenocarcinoma; uterine cancers including but not limited to, endometrial carcinoma and uterine sarcoma; ovarian cancers including but not limited to, ovarian epithelial carcinoma, borderline tumor, germ cell tumor, and stromal tumor; esophageal cancers including but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including but not limited to hepatocellular carcinoma and hepatoblastoma; gallbladder cancers including but not limited to, adenocarcinoma; cholangiocarcinomas including but not limited to, pappillary, nodular, and diffuse; lung cancers including but not limited to, non-small cell lung cancer, squamous cell carcinoma (epidermoid carcinoma), adenocarcinoma, large-cell carcinoma and small-cell lung cancer testicular cancers including but not limited to, germinal tumor, seminoma, anaplastic, spermatocytic, nonseminoma, embryonal carcinoma, teratoma carcinoma, choriocarcinoma (yolk-sac tumor); prostate cancers including but not limited to, adenocarcinoma, leiomyosarcoma, and rhabdomyosarcoma; penile cancers; oral cancers including but not limited to, squamous cell carcinoma; basal cancers; salivary gland cancers including but not limited to, adenocarcinoma, mucoepidermoid carcinoma, and adenoidcystic carcinoma; pharynx cancers including but not limited to, squamous cell cancer, and verrucous; skin cancers including but not limited to, basal cell carcinoma, squamous cell carcinoma and melanoma, and superficial spreading melanoma, nodular melanoma, lentigo malignant melanoma, acral lentiginous melanoma; kidney cancers including but not limited to, renal cell cancer, renal cancer, adenocarcinoma, hypemephroma, fibrosarcoma, and transitional cell cancer (renal pelvis and/or uterer); Wilms' tumor; bladder cancers including but not limited to, transitional cell carcinoma, squamous cell cancer, adenocarcinoma, and carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cvstadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In one embodiment, the cancer is benign, e.g., polyps and benign lesions. In other embodiments, the cancer is metastatic, hetIL-15 and ACT can be used in the treatment of pre-malignant as well as malignant conditions. Pre-malignant conditions include hyperplasia, metaplasia, and dysplasia. Treatment of malignant conditions includes the treatment of primary as well as metastatic tumors. In a specific embodiment, the cancer is melanoma, colon cancer, renal cell carcinoma, or lung cancer (e.g., non-small cell lung cancer). In certain embodiments, the cancer is metastatic melanoma, metastatic colon cancer, metastatic renal cell carcinoma, or metastatic lung cancer (e.g., metastatic non-small cell lung cancer).

### Examples

### Material and Methods

### Subcutaneous Mouse Tumor Model

B16F10 melanoma cells were maintained in DMEM supplemented with 10% heat-inactivated fetal bovine serum (FBS, ThermoFisher, Waltham, N.Y.) and penicillin/streptomycin. Before injection, B16 cells were washed twice and resuspended in DMEM without serum and antibiotics. Seven-week old wild type C57BL/6 animals were injected with 4×10⁵ tumor cells subcutaneously (SC) in the flank. MC38 colon carcinoma cells were maintained in DMEM supplemented with 10% heat-inactivated fetal bovine serum (FBS, ThermoFisher, Waltham, N.Y.), 1× penicillin/streptomycin, 1× essential amino acids and 1×HEPES. In some experiments, wild type C57BL/6 animals were injected with 4×10⁵ B16 melanoma cells SC into one flank and with 3×10⁵ MC38 colon carcinoma cells SC into the other flank. Tumor area (length×width) was measured every 2-3 days.

### Immunotherapy of B16 Melanoma Bearing-Mice

Five days after inoculation of B16 cells, tumor-bearing mice were randomized into three groups receiving ACT, ACT+XRT or ACT+hetIL-15. In some experiments, mice received ACT+IL-2. Splenocytes from pmel-1 TCR/Thy1.1 transgenic mice were harvested and used as the source of melanoma antigen (hgp100₂₅₋₃₃)-specific T cells (Pmel-1 T cells) for ACT. Single cell suspensions were generated from spleen. Splenocytes were then cultured using plates coated with anti-CD3 antibody (145-2C11, BD Bioscience, Frankin Lakes, N.J.) and soluble No Azide/Low Endotoxin (NA/LE) anti-CD28 antibody at 1 μg/ml (37.51, BD Bioscience) for in vitro activation. Fresh media supplemented with human IL-2 (12.5 ng/ml, Peprotech, Rocky Hill, N.J.) was provided on day 2 and cells were harvested and counted on day 5. All mice were injected intravenously with 1-5×10⁶ (in 100 μl PBS) of in vitro-activated Pmel-1 T cells in the absence of vaccination. For lymphodepletion preconditioning, mice were subjected to whole-body irradiation once (5 Gy; x-ray source, 1.29 Gy/min, 137-cesium chloride irradiator) one day before ACT. For hetIL-15 treatment, lyophilized hetIL-15 protein (37, 39) was dissolved in water for injection. Mice received intraperitoneal injection of 3 μg (molar mass of IL-15) of hetIL-15 in 200 μl every 2 to 3 days for a total of 8 injections. For the IL-2 treatment, mice received intraperitoneal injection of 3 or 9 μg of human IL-2 (Teceleukin, Hoffman-Roche) three times per week for 8 total injections. For the analysis of tumor-infiltrating lymphocytes, two independent experiments were performed using 5×10⁶ Pmel-1 cells for ACT. One experiment was performed using 1×10⁶ Pmel-1 cells for ACT, leading to similar results.

Isolation of Lymphocytes (TILs) from Tumor and Lymphoid Organs

Excised tumors were cut into small pieces and treated with collagenase IV (200 U/ml, Sigma-Aldrich, St. Louis, Mo.) and DNase I (30 unit/ml, Roche Diagnostic GmbH, Mannheim, Germany) at 37° C. for 1 hour. Collagenase digestion was stopped by adding HBSS supplemented with 2 mM EDTA. After filtration through a 100 μm cell strainer (BD Bioscience), tumor cell suspensions were layered on 3 ml histopaque 1116 (Sigma-Aldrich) and centrifuged at 2,000 rpm for 20 minutes at room temperature. Enriched live cells were collected at the interphase between histopaque and medium. TILs were washed with PBS and stained with the Fixable Viability dye (ThermoFisher) for 30 minutes at 4° C., before surface and intracellular staining for flow cytometry analysis. Cells were recovered from lungs using the same collagenase and DNase I procedure. After treatment with collagenase and DNase I, cells recovered from lung were filtered through a 100 μm cell strainer, washed with PBS and stained for flow cytometry analysis. Cells were also obtained from spleens and inguinal lymph nodes for surface and intracellular staining for flow cytometry analysis.

### Assay of Intracellular Cytokine Production by TILs and Splenocytes

Single cell suspensions from tumor and inguinal lymph nodes were cultured in medium only or in presence of the hgp100₂₅₋₃₃ peptide (KVPRNQDWL, 1 μg/ml, NeoScientific, Woburn, Mass.) at 37° C. for 6 hours (tumor) or 12 hours (lymph nodes). The antibody anti-CD107a (1D4B, 1:50) in and GolgiStop (BD Bioscience) were added during culture. Cells were then harvested and stained for surface and intracellular markers.

### Flow Cytometry Analysis

After preparing single cell suspension from tumors and lymphoid organs, cells were washed with PBS supplemented with 0.5% FBS and 2 mM EDTA. Surface staining was performed by using antibodies to the following markers: CD3 (145-2C11), CD4 (RM4-5), CD8 (53-6.7), CD45 (30-F11), CD90.1 (OX-7), PD-1 (RMP1-30), CD107a (ID4B) (BD Bioscience; eBioscience. Inc., San Diego, Calif.; Biolegend, San Diego, Calif.). Adoptively transferred Pmel-1 T cells were identified as CD3⁺CD8⁺Thy1.1⁺ cells, while endogenous CD8⁺ T cells were identified as CD3⁺CD8⁺Thy1.1⁻. For intracellular staining, cells were fixed and permeabilized using Foxp3 staining buffer (eBioscience, Inc.). Samples were stained with Ki-67 (SOLA15), granzyme B (GB11), T-bet (eBio4B10), and Foxp3 (FJK-16S) antibodies (eBioscience, Inc.). For intracellular cytokine staining, cells were fixed with BD fixation and permeabilization buffer (BD Bioscience). Samples were stained with antibodies to interferon-γ (XMG1.2) and tumor necrotic factor (MP6-XT22). After staining, cells were resuspended in 300 μl PBS supplemented with 0.5% FCS and analyzed in LSRII or LSRFortessa flow cytometers (BD Bioscience). All data analysis was performed using FlowJo software (Tree Star, Inc, Ashland, Oreg.).

### Immunohistochemistry

### Tissue Fixation and Processing

Tumors were harvested and fixed for 24 hours at room temperature (RT) in Zinc-Fixation Buffer. Tumor sections were paraffin embedded using Tissue-Tek automated tissue processor (Sakura) and embedded with Leica tissue embedder. Slides containing sections of 4.5 μm in thickness were then prepared.

### Deparaffinization, Staining, Imaging and Analysis

Slides were placed onto staining rack in a Leica autostainer and deparaffinized. Slides were treated with PeroxAbolish (Biocare Medical) for 20 min to reduce endogenous peroxidase activity, rinsed once with water, once with TBS-T and blocked with goat serum (Vector Labs) for 20 minutes. Rabbit anti-CD3 antibody (SP7, Spring Bioscience, M3074) was diluted 1:100 in Renaissance antibody diluent (Biocare Medical), added to slide and incubated for 45 minutes on an orbital shaker at room temperature. Slides were washed 3× for 30s in TBS-T. Anti-rabbit HRP secondary antibody (Life Technologies, 87-9623) was added and incubated for 10 minutes RT, and subsequently washed 3× for 30s in TBS-T. Tyramidefluorophore reagent (PerkinElmer, NEL791001KT; Life Technologies, T20950) was added to slides at 1:100 dilution in Amplification plus buffer (PerkinElmer, NEL791001KT) and incubated for 10 minutes at RT; slides were washed 3× for 30s in TBS-T followed by one wash with water. Slides were treated with PeroxAbolish for 20 min to eliminate peroxidase activity. The same cycle was repeated for the rat anti-CD8 (53-6.7, BD Biosciences, 550281, 1:100) primary antibody followed by anti-rat HRP (Vector Labs, MP-7444-15) secondary antibody and tyramide-fluorophore reagent. Slides were washed with TBS-T and H20 followed by antibody stripping using antibody-stripping buffer (0. IM glycine (Sigma, G2879), pH10 using NaOH, 0.5% Tween for 10 minutes at RT. Slides were rinsed with TBS-T, blocked with goat serum and incubated 45 min with rat anti-CD4 (RM4-5, BD Biosciences, 550280) diluted 1:100 in Renaissance antibody diluent. Slides were washed 3× for 30s in TBS-T, incubated with anti-rat HRP (Vector Labs, MP-7444-15) secondary antibody and tyramidefluorophore reagent. Slides were washed 3× for 30s in TBS-T followed by one wash with water. Slides were treated with PeroxAbolish for 20 min, washed IX with H20 and IX with TBS-T. Slides were incubated for 45 min with rat anti-CD90.1 PE (HIS51, eBioscience, 12-0900-81) diluted 1:50 in Renaissance antibody diluent. Slides were washed 3× for in TBS-T. Anti-PE HRP (KPL, 04-40-02) diluted 1:50 in Renaissance antibody diluent was added to slides and incubated for 10 minutes at RT. Slides were washed 3× with TBS-T and Tyramide-fluorophore reagent was added to slides at 1:100 dilution in Amplification plus buffer for 10 minutes at RT. Slides were rinsed in TBS-T, DAPI (Life Technologies, D1306, 1 mg/mL stock) was diluted 1:500 in PBS and added to slides. Slides were incubated for 5 minutes at RT, washed twice for 30s in TBS-T, were rinsed with water and coverslipped. Imaging at both 4× and 20× was performed using Vectra imaging software (PerkinElmer). The number of cells were enumerated from fifteen 20× fields using inForm analysis software (PerkinElmer).

### Statistics

Differences among groups were evaluated by One-Way analysis of variance ANOVA or unpaired student's t test. The p-values were corrected for multiple comparisons using Holm-Sidak test. Tumor growth over time was analyzed using repeated measures ANOVA after appropriate transformation of raw tumor area values to be consistent with the assumptions of the method. Prism 6.0c software package (GraphPad Software, Inc., La Jolla, Calif.) was used for analysis.

### Results

Adoptively Transferred Pmel-1 Cells Infiltrate and Persist in Tumor Sites Upon hetIL-15 Administration

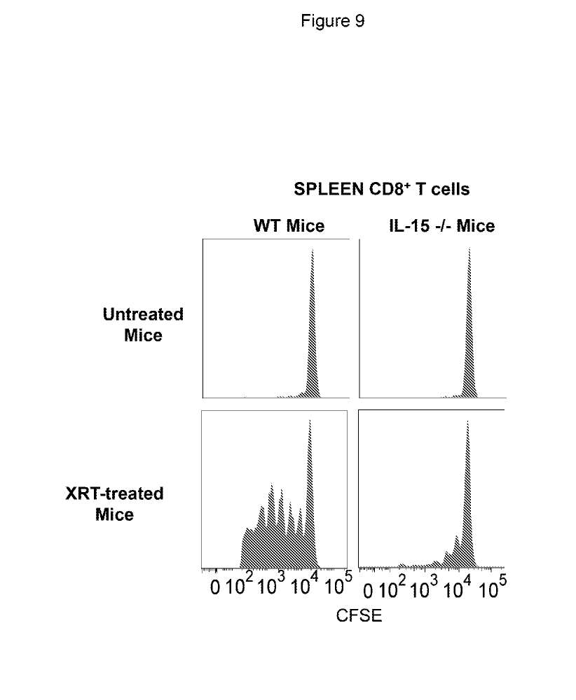

Previous studies have shown that the increased availability of homeostatic cytokines following lymphodepleting treatment of the host sustains the proliferation of adoptively transferred cells and results in significantly improved ACT therapy outcomes for cancer (11). In view of these studies, the behavior of CFSE-labeled transferred CD8-T cells in wild type as well as in IL-15KO mice was analyzed. It was confirmed that lack of IL-15 partially eliminated the proliferation of adoptively transferred cells, suggesting that IL-15 is a non-redundant factor participating in this process (FIG. 9).

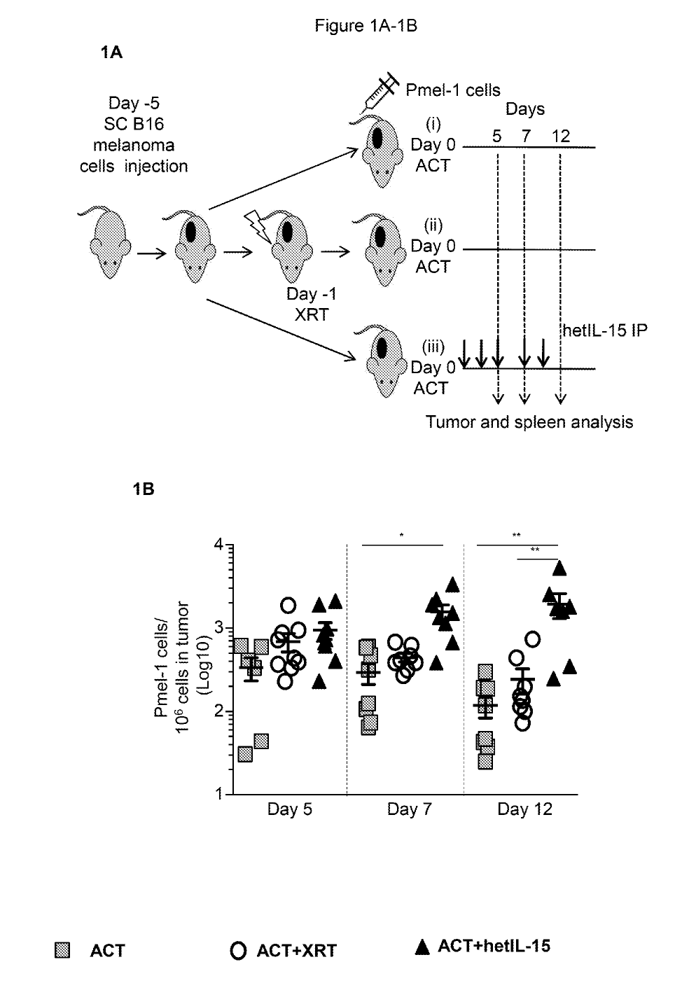

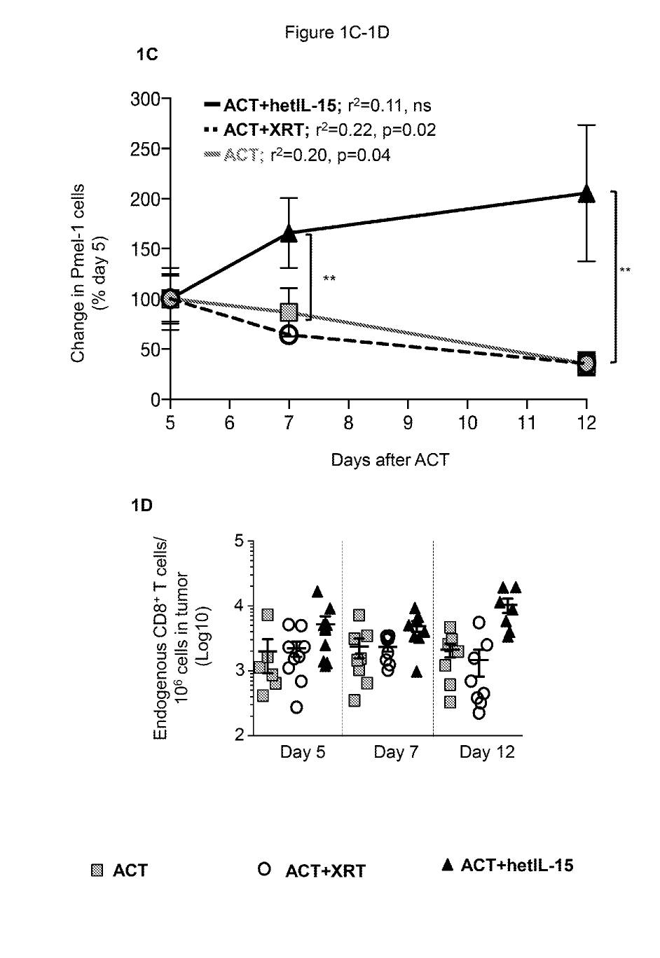

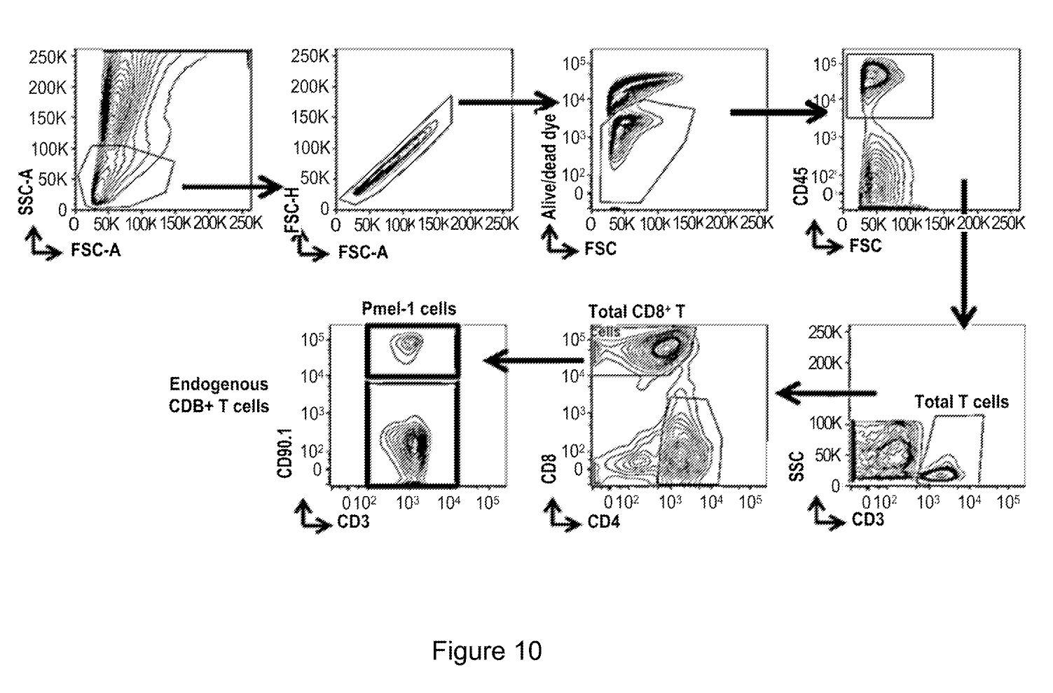

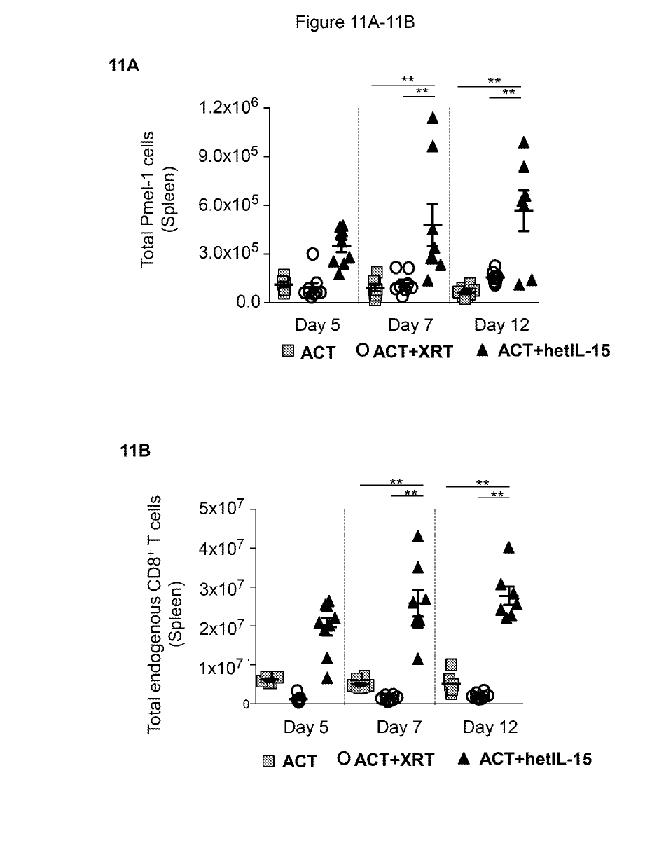

Studies were then conducted to determined whether exogenous hetIL-15 administration could overcome endogenous cellular “sinks”, normally competing for access to IL-15, to sustain ACT in the absence of lymphodepletion. For this analysis, the B16 melanoma mouse model was selected, where tumor cells express the melanoma cell-associated antigen gp100. For ACT therapy. C57BL/6-pmel-1-Thy1.1 transgenic mice were used as a source of Pmel-1 cells. These cells have a T-cell receptor that specifically recognizes the gp100₂₅₋₃₃ peptide (38). Pmel-1 cells were transferred into B16 melanoma-bearing C57BL/6 mice comparing 3 strategies (FIG. 1A): (i) cell transfer without lymphodepletion (ACT), (ii) cell transfer in irradiated mice (ACT+XRT) and (iii) cell transfer plus exogenous hetIL-15 administration (ACT+hetIL-15) in lymphoreplete mice. Tumor infiltration of adoptively transferred Pmel-1 cells as well as endogenous CD8⁺ T cells was measured over time. Tumors were isolated at specified time points and the tumor-infiltrating lymphocytes (TILs) were analyzed by flow cytometry as detailed in FIG. 10. Tumor-infiltrating Pmel-1 cells were distinguished from endogenous CD8+ T cells by the expression of CD90.1. In the ACT group, ˜300 Pmel-1 cells per million cells were present at the tumor site at day 5 after cell transfer (FIG. 1B). At the same time point, a slight increase (˜2-3 fold) in the proportion of Pmel-1 cells in tumor was detected both in mice pre-treated with XRT or in mice receiving hetIL-15, although this difference did not achieve statistical significance (one-way Anova analysis) (FIG. 1B). Importantly, in both the ACT and ACT+XRT groups, a progressive decline in the frequency of Pmel-1 cells in the tumor was observed. The number of Pmel-1 cells per million cells in the tumor decreased by approximately 60% between day 5 and day 12 after cell transfer (FIGS. 1B&C). These results suggest that irradiation supports the infiltration of tumor by tumor-specific Pmel-1 T cells, but provides only limited benefits for their persistence in situ. In contrast, tumor-bearing mice that received hetIL-15 in combination with ACT in the absence of lymphodepletion showed persistence of Pmel-1 cells in the tumor. These Pmel-1 cells were still present at high number (2000 Pmel-1 cells per million cells in the tumor) at day 12 after cell transfer (FIGS. 1B&C). Therefore, administration of hetIL-15 in the absence of lymphodepletion favors both infiltration and persistence of antigen-specific transferred cells in the tumor.

The effects of hetIL-15 treatment on tumor infiltration by endogenous CD8⁺ T cells was further investigated. Administration of hetIL-15 also significantly increased the frequency of tumor-resident CD8⁺ T cells in comparison to both ACT and ACT+XRT groups at day 12 after ACT (FIG. 1D). Comparison of the ACT and ACT+XRT groups showed no difference in the number of endogenous CD8+ T cells in the tumor (FIG. 1D).

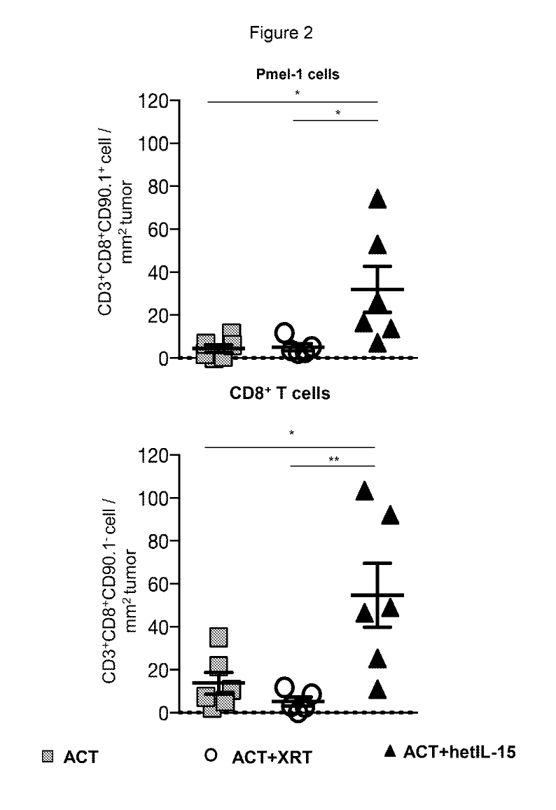

To confirm that the lymphocytes isolated upon in vitro digestion of the tumor were of intratumoral origin rather than peripherally associated with excised tumors, the infiltration of T cells was evaluated using fluorescence immunohistochemistry. Staining of tumor sections at day 13 after cell transfer was performed using antibodies against CD3, CD4, CD8 and CD90.1. The staining results confirmed that, in comparison to the other groups, treatment with hetIL-15 resulted in an increased accumulation of both tumor specific Pmel-1 cells and endogenous CD8⁺ T cells that were spread throughout the tumor area. The quantitation of Pmel-1 cells and endogenous CD8⁺ T cells/mm² by fluorescence immunohistochemistry showed a significant increase in the ACT+hetIL-15 group (FIG. 2). Taken together, these data support the conclusion that IL-15 promotes the infiltration of tumor sites by both adoptively transferred antigen-specific T cells and by endogenous CD8+ T cells and favors their in situ persistence in the absence of lymphodepletion.

hetIL-15 Administration Promotes Preferential Enrichment of Pmel-1 Cells in Tumors in an Antigen-Dependent Manner

Whether hetIL-15 treatment differentially affects tumor-specific Pmel-1 cells and endogenous CD8⁺ T cells in a tumor compared to other tissues lacking gp100, i.e., spleen, lung and gp100-negative tumor (i.e., MC38 colon carcinoma) was then investigated. Similarly to the findings in tumor, hetIL-15 administration in the absence of lymphodepletion resulted in a significant increase in the total count of both Pmel-1 cells (FIG. 11A) and endogenous CD8+ T cells (FIG. 11B) in spleen, in comparison to both ACT and ACT+XRT.

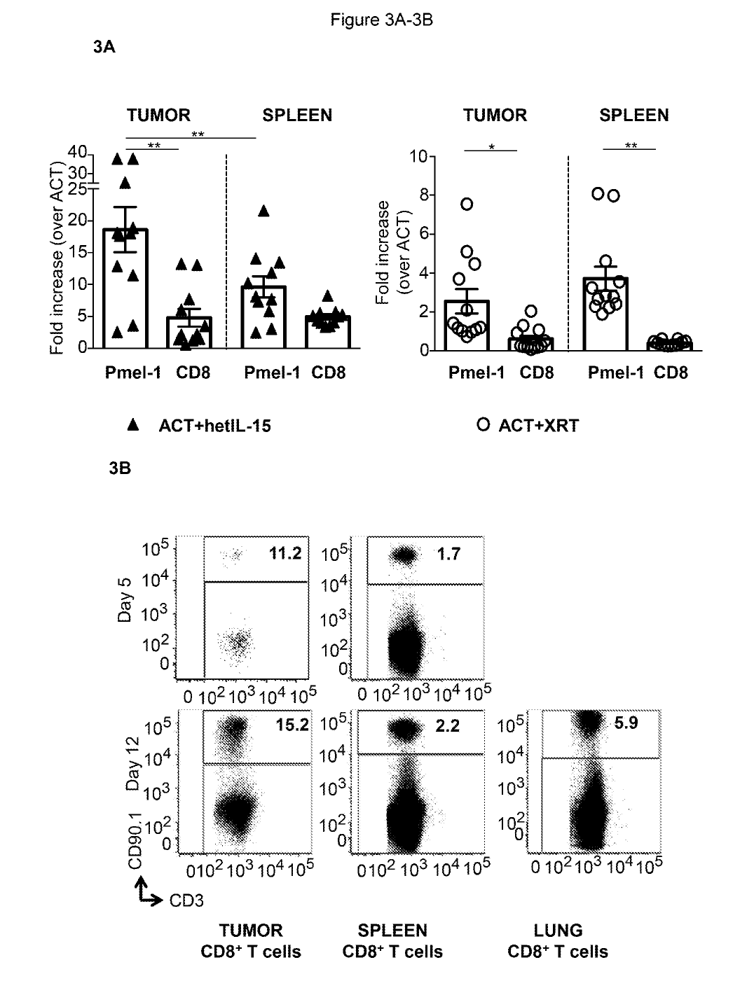

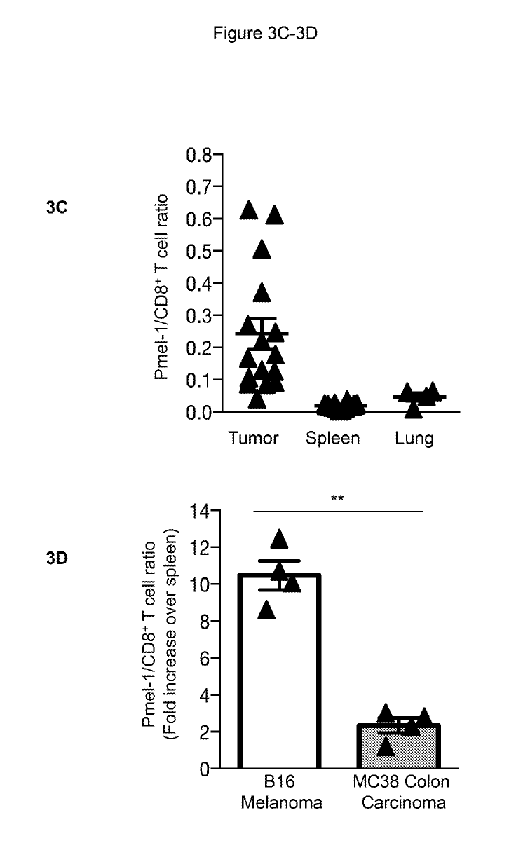

Interestingly, in comparison to ACT alone, hetIL-15 administration resulted in a proportionally greater enrichment of Pmel-1 cells than endogenous CD8+ T cells in tumor, as shown by both flow cytometry (FIG. 3A left panel, day 12 after cell transfer) and immunohistochemistry (day 13 after cell transfer, data not shown) analyses. In contrast, hetIL-15 treatment induced similar changes in Pmel-1 cells and endogenous CD8⁺ T cells in spleen. In addition, while the hetIL-15-dependent expansion of CD8+ T cell was comparable in tumor and spleen, hetIL-15 preferentially increased Pmel-1 cells resident in tumor than the same population in spleen (FIG. 3A, left panel). In the group of mice pre-treated with XRT, Pmel-1 cells were also significantly enriched in comparison to endogenous CD8⁺ T cells in the tumor, but Pmel-1 cells were equally affected by the treatment in both tumor and spleen (FIG. 3A, right panel). These results suggest that, upon hetIL-15 treatment, the accumulation of Pmel-1 cell was not just a consequence of the generalized IL-15-driven effects on the whole CD8⁺ population but, rather, that hetIL-15 regulates TILs and adoptively transferred cells in an antigen-specific manner.

The frequency of Pmel-1 cells within the CD8⁺ T cell population as well as the Pmel-1/CD8⁺ T cell ratio in different organs was then investigated. In mice that received ACT+hetIL-15, ˜10-15% of CD8⁺ T cells infiltrating the tumor were Pmel-1 cells in comparison to ˜2% in spleen (FIG. 3B), resulting in an approximately 10-fold increase in the Pmel-1/CD8⁺ T cell ratio in B16 tumor in comparison to spleen (FIGS. 3C&D). To rule out a generalized IL-15-dependent mobilization of transferred cells to effector sites, the effect of hetIL-15 on both Pmel-1 and endogenous CD8+ T cells in lung was evaluated. The results showed that upon hetIL-15 administration, only ˜5% of CD8⁺ T cells infiltrating the lung were Pmel-1 cells (FIG. 3B), and the Pmel-1/CD8+ T cells ratio in lung was similar to the one observed in spleen (FIG. 3C).

Next, the dependency of infiltration and persistence of Pmel-1 cells in tumor areas upon gp100 antigen was then evaluated. Mice were co-injected with gp100⁺ B16 melanoma cells and gp100⁻ MC38-colon carcinoma cells. The Pmel-1/CD8⁺ T cells ratio was determined in both the B16 and MC38 tumors as well as in spleen. The results showed that Pmel-1 specifically localized to the B16 tumor, while the infiltration of MC38 colon carcinoma by Pmel-1 cells was similar to that of spleen and lung (FIG. 3D). Taken together, these data suggest that hetIL-15 administration promotes a persistent and antigen-dependent enrichment of transferred tumor-specific T cells in the tumor sites in comparison to lymphoid and non-lymphoid organs.

hetIL-15 Administration Supports Effector Functions of Transferred Tumor-Infiltrating Pmel-1 Cells.

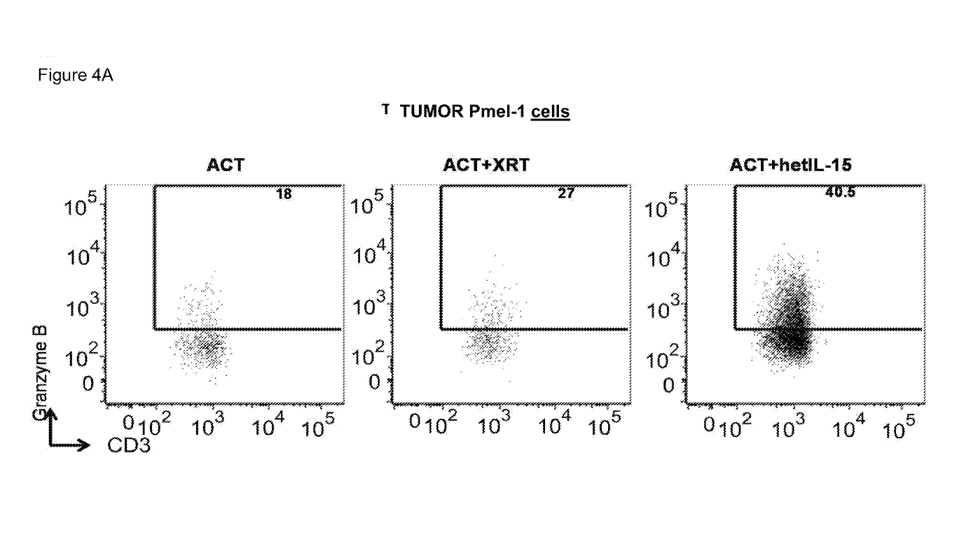

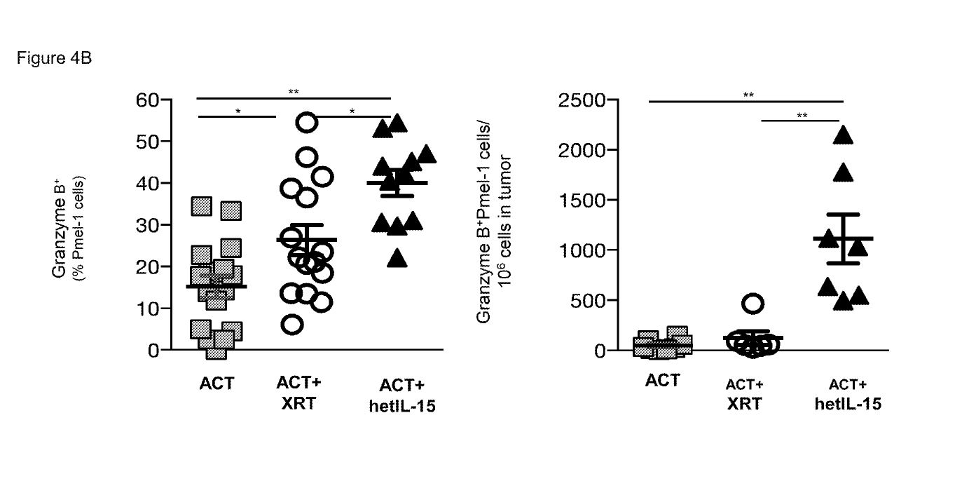

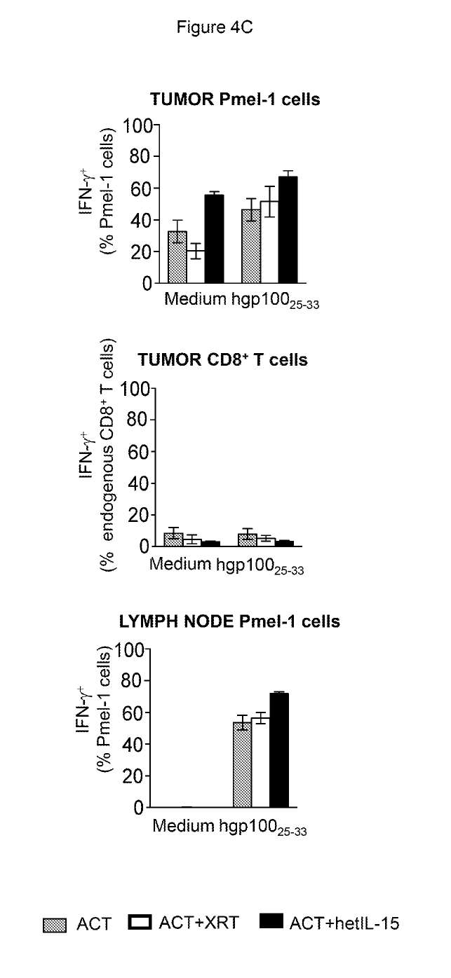

The functional competency of tumor-resident Pmel-1 was then evaluated. IL-15 has been reported to play a pivotal role in stimulating the killing activity of lymphocytes, through the up-regulation of the cytotoxic molecule granzymne B (GzmB) (40, 41). Intracellular staining followed by flow cytometry was used to assess the frequency of Pmel-1 cells in the tumor expressing GzmB (FIGS. 4A&B). Irradiation pre-conditioning resulted in a significant increase in the percentage of tumor-resident GzmB⁺ Pmel-1 cells in comparison to ACT regimen only, suggesting that the irradiation generated an environment supporting the killing activity of transferred cells. Moreover, providing hetIL-15 in absence of lymphodepletion resulted in the highest proportion of tumor-infiltrating GzmB⁺ Pmel-1 cells (FIGS. 4A&B). This led to a significant increase in the total number of GzmB+ Pmel-1 cells per tumor upon hetIL-15 administration (FIG. 4B, right panel), which was superior to the other treatments.

The production of IFN-γ by adoptively transferred Pmel-1 cells was also investigated. In all treatment groups, tumor resident Pmel-1 cells were characterized by the ability to secrete IFN-γ upon ex vivo culture in the absence of stimulation. This is likely the result of the stimulation of Pmel-1 cells by the presence in the single cell suspension of tumor cells expressing the gp100 antigen. Under this condition, a significantly greater proportion of IFN-γ Pmel-1 cells was found in mice in the ACT+hetIL-15 group (FIG. 4C, left panel), suggesting that hetIL-15 increases the frequency of adoptively transferred cells producing IFN-γ in the tumor. Upon stimulation of ex vivo cultures with hgp100₂₅₋₃₃ peptide, all three treatment groups showed an increase in the frequency of Pmel-1 cells producing IFN-γ and no statistical difference was found among the groups (FIG. 4C, left panel). As control, we also evaluated the proportion of endogenous CD8⁺ T cells producing IFN-γ in ex vivo 6-hour cultures of dissociated tumors. In all the groups, less than 10% of endogenous CD8⁺ T cells secrete IFN-γ, and this frequency did not change upon hgp100₂₅₋₃₃ peptide stimulation (FIG. 4C, middle panel). A similar analysis was also performed on total lymphocytes isolated from lymph nodes of treated mice. In the absence of stimulation, Pmel-1 cells did not secrete IFN-γ, suggesting that the tumor lymphocyte response described above in the absence of peptide stimulation is antigen-specific. Stimulation of lymph node lymphocytes with the hgp100₂₅₋₃₃ peptide induced an IFN-γ response in all groups. Mice receiving ACT+hetIL-15 treatment showed a significantly higher frequency of IFN-γ⁺Pmel-1 cells in comparison to the other treatments (FIG. 4C, right panel). Overall, these data indicate that hetIL-15 treatment sustains the cytotoxic potential and the ability to produce IFN-γ of adoptively transferred cells in the absence of lymphodepletion.

hetIL-15 Administration Decreased PD-1 Levels on Tumor-Infiltrating Pmel-1 Cells, while Sustaining their Proliferation and Cytotoxic Functions

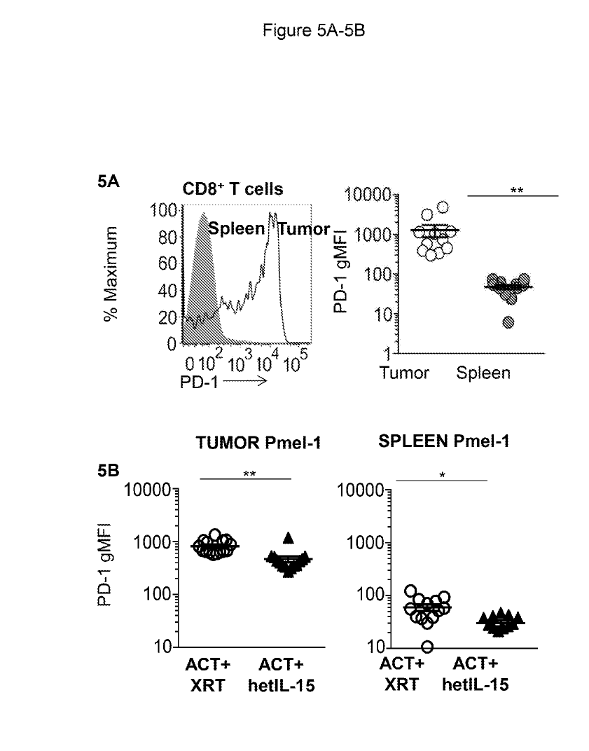

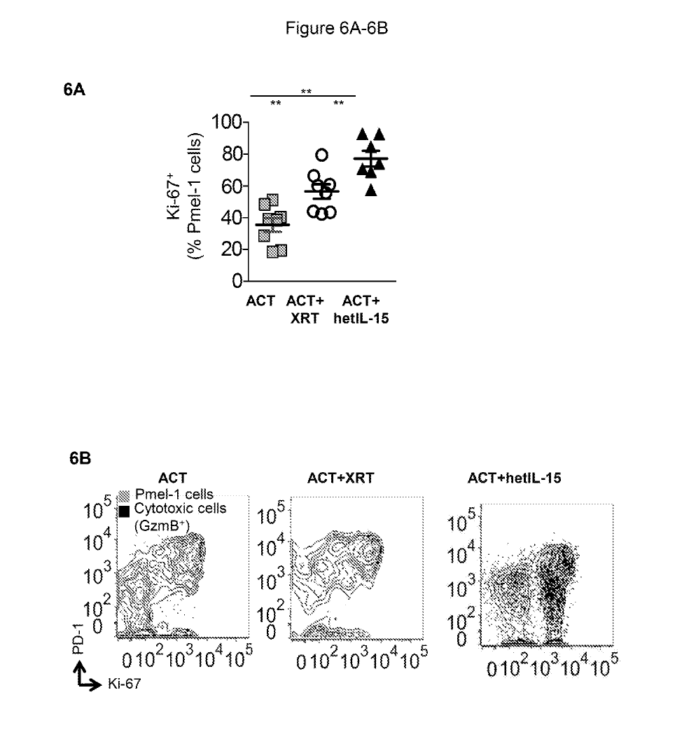

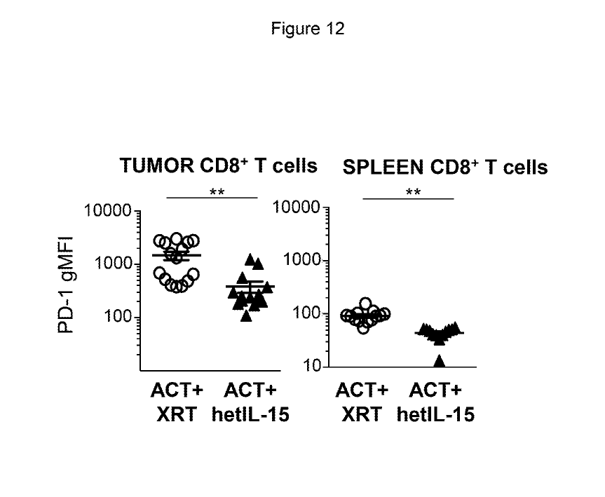

The tumor microenvironment is immunosuppressive and can be characterized by high levels of negative regulators, such as PD-1/PD-L1 (41-44). Indeed, in untreated B16 melanoma-bearing mice, the endogenous CD8 T cell population exhibited significantly higher PD-1 levels in the tumor environment (FIG. 5A, black) in comparison to the spleen (FIG. 5A, solid grey). In comparison to the ACT+XRT group, hetIL-15 treatment significantly decreased the intensity of PD-1 expression per cell on both tumor-infiltrating Pmel-1 (FIG. 5B) and endogenous CD8⁺ T cells (FIG. 12) in the tumor and spleen.

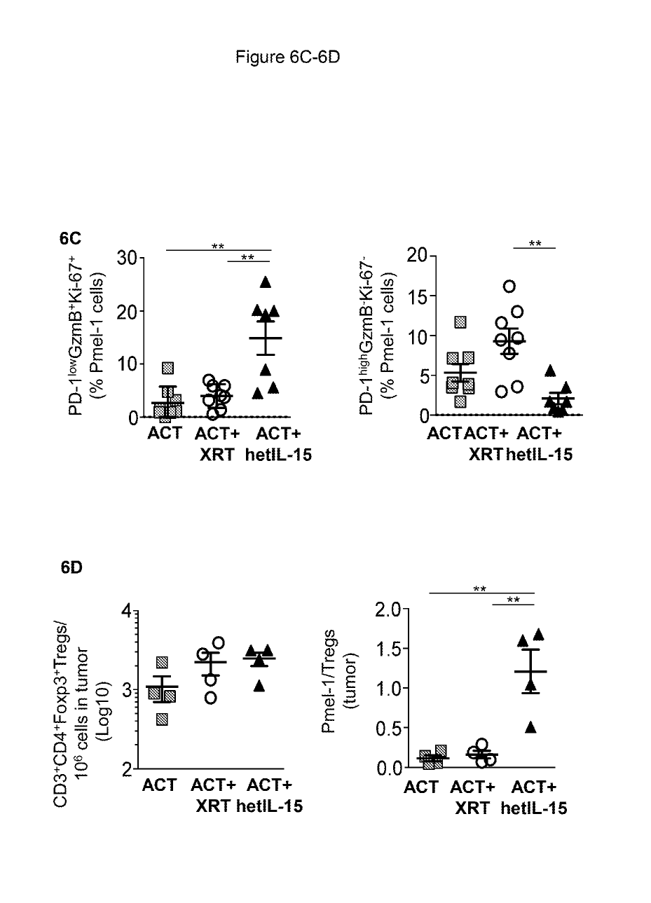

Interestingly, in comparison to ACT alone, both the ACT+XRT and ACT+hetIL-15 treatments increased the frequency of tumor-infiltrating Pmel-1 cells expressing the proliferative marker Ki-67, with hetIL-15 administration resulting in a higher frequency of proliferating tumor infiltrating Pmel-1 cells (FIG. 6A and Table 1). In the ACT+XRT regimen group, tumor proliferating Pmel-1 cells were characterized by higher level of PD-1, suggesting a more “exhausted” phenotype (FIG. 6B). In contrast, treatment with hetIL-15 resulted in a significant increased tumor accumulation of a population of proliferating Pmel-1 cells with a lower level of PD-1 expression (FIG. 6B). These cells were also the main producers of GzmB and represented ˜15% of the whole Pmel-1 population resident in the tumor (versus ˜5% in the ACT alone and ACT+XRT groups, FIG. 6C, left panel). hetIL-15 administration also resulted in a significantly reduced tumor frequency of Pmel-1 cells with the exhaustion-like phenotype GzmB-Ki67-PD-1 high, supporting a role for IL-15 in rescuing cells from exhaustion (FIG. 6C, right panel). The proportion of the different subsets of tumor-infiltrating Pmel-1 cells analyzed for the expression of Ki67, GzmB and PD-1 is depicted in Table 1. These data suggest that hetIL-15 treatment alleviates the exhaustion of adoptively transferred T cells infiltrating the tumor while sustaining their proliferative and effector functions.

TABLE 1
PD-1, Ki67 and GzmB expression by tumor-infiltrating Pmel-1 cells.
Pmel-1 cells infiltrating the tumor were analyzed for the expression
of PD-1, Ki67, and GzmB by flow cytometry. The percentage of the
different tumor-infiltrating Pmel-1 subsets is shown. The analysis
was performed at day 12 after ACT. Combined data from two
independent experiments are shown.

| | | | ACT + |
|---|---|---|---|
| | ACT | ACT + XRT | hetII-15 |
| GzmB⁺Ki67⁺PD-1^{low} | 2.7 | 4 | 15.2 |
| (% total Pmel-1) | | | |
| GzmB⁻Ki67⁻PD-1^{high} | 5.4 | 9.4 | 2.1 |
| (% total Pmel-1) | | | |
| GzmB⁻Ki67⁺PD-1^{high} | 9.5 | 14.4 | 11.6 |
| (% total Pmel-1) | | | |
| GzmB⁺Ki67⁻PD-1^{high} | 1.5 | 3.1 | 0.6 |
| (% total Pmel-1) | | | |
| GzmB⁺Ki67⁺PD-1^{high} | 4.1 | 5.9 | 4.8 |
| (% total Pmel-1) | | | |
| GzmB⁻Ki67⁻PD-1^{low} | 59.7 | 41.5 | 22.4 |
| (% total Pmel-1) | | | |
| GzmB⁻Ki67⁺PD-1^{low} | 10.4 | 12.5 | 35.9 |
| (% total Pmel-1) | | | |
| GzmB⁺Ki67⁻PD-1^{low} | 6.6 | 9.3 | 9.7 |
| (% total Pmel-1) | | | |

hetIL-15 Increases the Ratio of Pmel-1 Cells to Treg in Tumors

The effects of hetIL-15 administration on tumor-resident CD4+Foxp3+ Treg cells was also evaluated. Analysis at day 12 after ACT showed no difference in the number of Tregs per million cells present at the tumor sites among the three groups (FIG. 6D, left panel), suggesting that hetIL-15 does not significantly impact the frequency of Tregs in the tumor. Favorable cancer immunotherapy treatments have been previously linked to the ratio of CD8+ T cells to Tregs (97,98). For this purpose, we determined the Pmel-1/Treg ratio within the tumor after ACT. Tumors of mice that received either ACT alone or ACT+XRT were characterized by a Pmel-1/Treg ratio of ˜4.2, showing that Treg cells largely outnumber tumor-specific adoptively transferred cells. Due to its positive effect on the persistence of Pmel-1 cells, hetIL-15 administration resulted in a ˜10× increase in the Pmel-1/Treg ratio within the tumor, in comparison to both ACT and ACT+XRT (FIG. 6D, right panel).

Overall, these data indicate that hetIL-15 does not significantly affect the frequency of tumor-resident Treg and promotes an increased Pmel-1/Treg ratio within the tumor.hetIL-15 promotes tumor control and increased survival after ACT.

hetIL-15 Promotes Tumor Control and Increased Survival after ACT

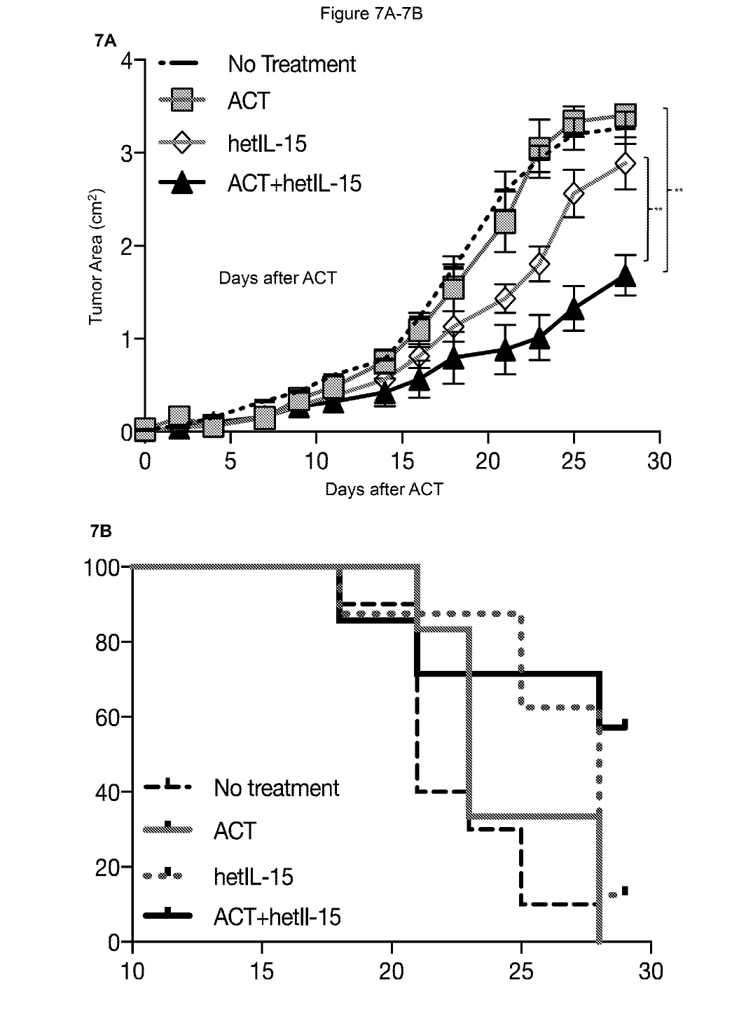

Given the effects of hetIL-15 administration in the absence of lymphodepletion on transferred tumor-specific T cells, the ability of this treatment to control tumor growth was evaluated. Monotherapy with IL-15 has been reported to promote tumor control in several murine cancer models (45-50). Indeed, in B16-melanoma bearing mice, eight administrations of hetIL-15 IP every 2 days resulted in a significant delay in tumor growth in comparison to PBS-treated mice (FIG. 7A). The anti-tumor potential of ACT alone or in comparison to ACT+hetIL-15 was also investigated. In the absence of lymphodepletion pre-conditioning and vaccination post-injection (51), ACT alone was ineffective, while the addition of hetIL-15 to ACT resulted in a significant improvement in tumor control in comparison to both ACT only- and IL-15 only-treated animals (FIG. 7A). All animals that received either PBS or ACT alone were sacrificed within 5 weeks after tumor injection due to a large tumor mass. However, at the same time point, the survival rate in the hetIL-15+ACT group was 60% (FIG. 7B). These data indicate that administration of hetIL-15 can improve treatment outcomes of ACT without the use of potentially toxic host immune depletion prior to cell infusion.

### IL-2+ACT Regimen Sustained Tumor Accumulation of Both Pmel-1 and Treg Cells

IL-2, like IL-15, is a member of the γ-chain family of cytokines. IL-2 is used as a clinically available cytokine for growing lymphocytes. This example compares the effects of IL-15 to IL-2 in combination with ACT in absence of lymphodepletion. To this purpose, B16-bearing mice were randomized into 3 groups receiving the following treatments: ACT alone, ACT+hetIL-15 and ACT+IL-2. Despite the toxicity reported in clinical studies, we verified that treatment with IL-2 is well tolerated in mice. A trend toward an increase in WBC and lymphocyte counts comparable to the ones induced by hetIL-15 was observed at day 12 after ACT, and no other hematologic changes were observed.

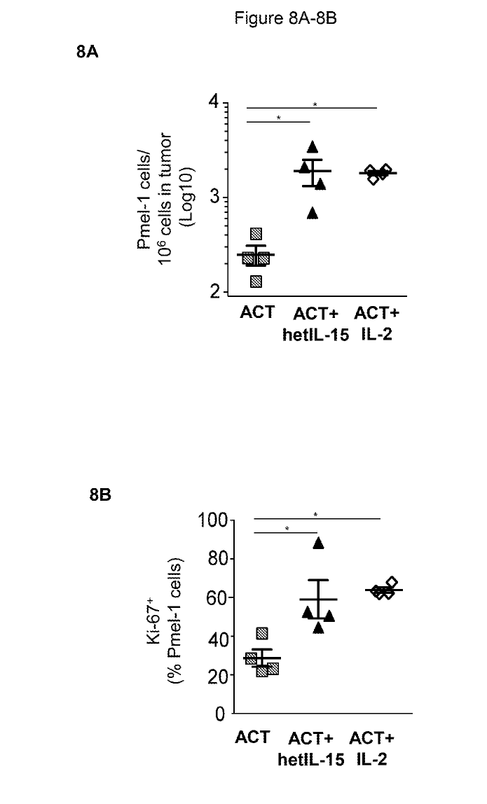

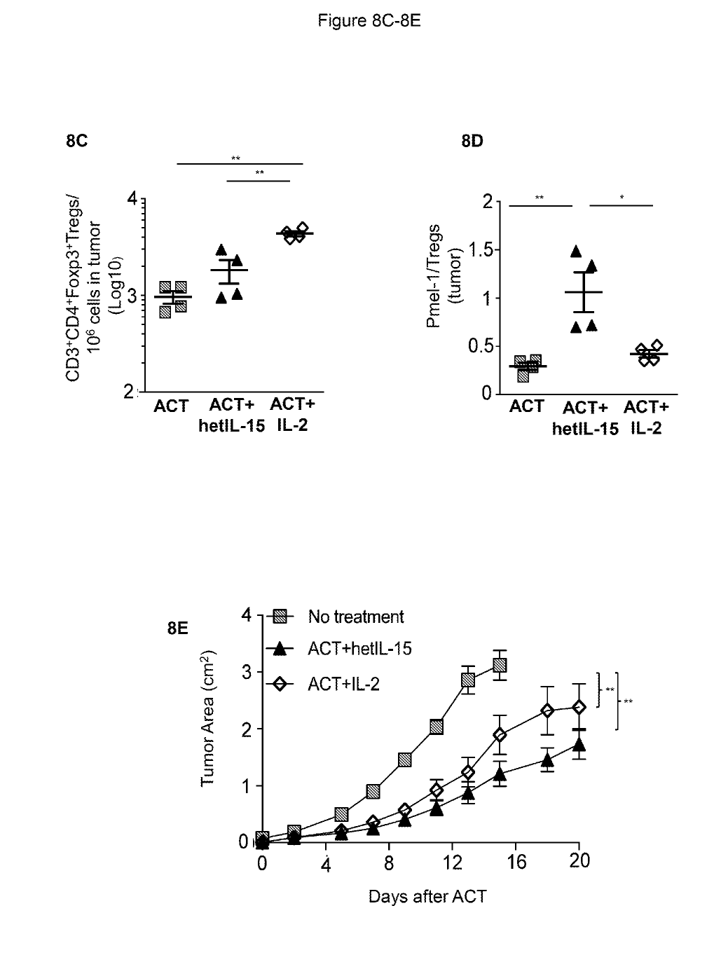

Tumors were isolated at day 10 after ACT and TILs were analyzed by flow cytometry. In agreement with the results presented in FIG. 1B, hetIL-15 induced a ˜10× increase in the accumulation of Pmel-1 cells at tumor sites, in comparison to mice that received ACT alone. Administration of IL-2 in the absence of irradiation resulted in a similar accumulation of tumor-infiltrating Pmel-1 cells (FIG. 8A). Functional analysis of tumor-infiltrating Pmel-1 cells showed that both cytokines induced a similar frequency of proliferating Ki6T7Pmel-1 that was significantly higher than animals receiving ACT alone (FIG. 8B).

IL-2 is the main growth factor for Treg in vivo. Upon IL-2 administration, the frequency of Treg within the tumor increased significantly in comparison to both ACT and ACT+hetIL-15 (FIG. 8C). The Pmel-1/Treg ratio within the tumor for the three treatment regimens was also determined. The positive effects of IL-2 on the tumor accumulation of both Pmel-1 cells and Tregs resulted in a Pmel-1/Tregs ratio 0.3, similar to the one observed in animals that received ACT alone (FIG. 8D). In contrast, hetIL-15 resulted in an increased Pmel-1/Treg ratio (˜1) (FIG. 8D), as also concluded above (FIG. 6D).

Additional experiments were conducted to show how these regimens compared in the control of tumor growth. Both cytokines were effective in inducing a significant delay in tumor growth, in comparison to untreated animals (FIG. 8E). In comparison to IL-2, hetIL-15 showed a trend towards better tumor control.

These data thus indicated that the γ-chain family of cytokines IL-2 and hetIL-15 can be beneficial in supporting ACT without irradiation, but that hetIL-15 has the additional advantage of preventing Treg accumulation.

### Summary of Examples

The illustrative data indicate that hetIL-15 administration in combination with adoptive cell transfer can enhance antitumor treatment efficacy in the absence of lymphodepletion. This cancer immunotherapeutic protocol aims to replicate the advantages of lymphodepletion preconditioning of the host for successful ACT while avoiding the potential adverse effects associated with lymphodepletion, including bacterial and opportunistic infections, needs for transfusions, and renal insufficiency (10, 52).

The non-redundant role of IL-15 in the survival, proliferation and cytotoxic activity of lymphocytes is well-established (20-23). Due to its functions, IL-15 has promising applications in cancer immunotherapy, as several experiments in mice have demonstrated (45-50). IL-15, either as single-chain molecule produced in E. coli (53) or as mammalian-derived hetIL-15 (NCT02452268, (37)) is currently being evaluated in Phase I clinical trials in cancer patients. In these studies, IL-15 has been well tolerated and characterized by an acceptable toxicity profile in humans (53, 54). hetIL-15 is the natural and stable form of the cytokine and offers unique advantages over the single-chain molecule for clinical use (31, 33, 37).

Determinants for effective ACT therapy resulting in tumor rejection have been previously identified (51, 55). Successful ACT therapies typically involve the transfer of a high number of tumor-specific lymphocytes that are capable of infiltrating the tumor, persisting and proliferating in vivo (56-60). Additionally, anti-tumor T cells must maintain specific effector properties, such as the production of cytokines IFN-γ (56), IL-2 (61), and cytotoxic molecules (56). Several lines of evidence have linked the stemness phenotype of T cells with a greater degree of ACT therapy success (62-66). In addition to modulating the intrinsic properties of antitumor T cells, successful outcomes following ACT also require manipulation of the host. Indeed, host lymphodepletion by irradiation or chemotherapy has been incorporated into clinical protocols. However, these interventions pose serious risks to humans, such as inefficient T cell repertoire restoration and immune dysfunction (13-15). The illustrative data provided herein indicate that an ACT+hetIL-15 regimen resulted in increased infiltration and persistence of adoptively transferred cells in the tumor. Importantly, transferred T cells proliferated in situ and exhibited a cytotoxic phenotype, resulting in slower tumor growth. Overall, our study confirmed previous findings that lymphopenia is not a prerequisite for effective ACT (67). Several other approaches to improve ACT outcomes in the absence of irradiation and chemotherapy have been recently explored, including the use of antibodies for specific cell type depletion (67, 68), genetically engineered tumor-specific T cells (69, 70), Toll-like receptor (TLR) ligands (71) and other Y-chain cytokines (67, 72).

One foreseeable advantage of the regimen proposed in this study is the absence of CD4⁺ T cells ablation. Indeed, several studies have underlined the important contribution of these cells in tumor control, via both exerting direct cytotoxic effector functions and providing help to CD8+ T cells (73-77). IL-15 has been linked to the proliferation of effectors CD4+ T cells and to the induction of their cytotoxic phenotype (78, 79).

A major obstacle to successful cancer immunotherapy is overcoming the immunosuppressive environment of the tumor. Two major categories of immune resistance within the tumor microenvironment have been proposed, lack of tumor-infiltrating cytotoxic T cells and immune inhibitor pathways (80, 81). The experiments described herein provide data illustrating the effect of IL-15 treatment in overcoming tumor immune resistance by acting on both mechanisms.

The absence of inflammatory stimuli within the tumor microenviromnment results in poor release of chemokines and poor mobilization of cytotoxic T cells to the tumor (82). Weak inflammation within the tumor has been proposed as a biomarker for predicting a poor response to cancer immunotherapy (81). The results described above showed that hetIL-15 administration favors a pro-inflammatory environment, and adoptively transferred cells specifically infiltrate and proliferate in the tumor, in an antigen-specific way. Under this regimen, the frequency of Pmel-1 cells within the CD8⁺ T cells population and the Pmel-1/CD8+ T cells ratio was higher in B16 melanoma (gp100+ tumor), in comparison to another type of tumor (MC38 colon carcinoma) in the same mouse, or in comparison to lymphoid and non-lymphoid organs (spleen and lung) lacking gp100 expression. In particular, the analysis of Pmel-1 cells frequency in the lung allows evaluation of the effects of IL-15 in mobilizing the general CD8⁺ T cell population to effector sites. Analysis of the Pmel-1 frequency in gp100⁻ MC38 colon carcinoma may in addition account for effects related to the enhanced vascular permeability and retention in the tumor. It has previously been shown that, upon irradiation, adoptively transferred cells are characterized by a ubiquitous distribution in the body regardless of the expression of the specific antigen, while retaining their cytotoxic activity only against tumor cells (83). Our results agree with this conclusion since our measurements show that irradiation increased Pmel-1 cells in tumor and spleen to similar levels. Currently, cancer vaccination strategies can result in the development of specific anti-tumor T cells that fail to efficiently localize to tumor sites (84, 85). Our study suggests a role of IL-15 signaling in combination with the engagement of the TCR on CD8+ T cells by the specific antigen, to promote T cell migration, and increase specific contact with an antigenic tumor, leading to more efficient control. This also suggests the use of IL-15 in combination with innovative cancer vaccine strategies (86).

Inflammation within the tumor is often linked to tumor adaptation, consisting of an upregulation of immune-inhibitory pathways, which ultimately renders the infiltrating tumor-specific T cells nonfunctional. The presence of CD8⁺ T cells within the tumor result in the upregulation of PD-L1 by cancer cells through the production of IFN-γ and in increased frequency of Tregs through the release of CCR4-binding chemokines and induced proliferation (82). Indeed, a major breakthrough in cancer immunotherapy was achieved by the use of checkpoint inhibitors that alleviate the immuno-resistance of the tumor, either by deleting immunosuppressive cells such as Tregs or by reverting anergy/exhaustion of T cells (87-90). Several studies have identified a role for IL-15 in reverting anergy and in rescuing CD8 T cells for an effective response against cancer cells (28, 29). The illustrative experiments described herein demonstrated the downregulation of PD-1 on adoptively transferred cells both in tumor and spleen. The tumor enrichment of PD-1^{low}Pmel-1 cells upon hetIl-15 treatment may be a consequence of recent tumor infiltration by fresh PD-1^{low}Pmel-1 cells, or of a direct feedback mechanism. A possible direct effect of hetIL-15 in downregulating PD-1 on lymphocytes could be exerted through the transcription factor T-bet, which has been shown to downregulate directly PD-1 on T cells, through the specific binding to the promoter of pd-1 gene, in the context of chronic infectious disease (91). According to this hypothesis, IL-15 signaling promotes the expression of T-bet (92) facilitating both PD-1 downregulation and cytotoxic maturation of Pmel-1. Thus, hetIL-15 resulted in the tumor enrichment of fully functional effector cells (PD-1^{low}Ki67⁺GzmB⁺), while ACT+XRT promoted lower number of tumor-specific cells with a more exhaustion-like phenotype (PD-1^{high}Ki67⁻GzrmB⁻). These results indicate that T cell exhaustion status may be one limitation of irradiation/lymphodepletion for effective ACT.

Although hetIL-15 delayed tumor growth in our experiments, it did not completely eradicate rapidly growing B16 tumors. This could be the result of suboptimal dosing of hetIL-15 in these particular experiments, since we have observed that local injection of hetIL-15 in the area of MC38 tumors can completely block tumor growth and results in some regressing tumors (unpublished results). Further protocol optimization, more prolonged treatment with hetIL-15 and combination of hetIL-15 with therapeutic vaccination and/or checkpoint inhibitors warrant further investigation. The effects of hetIL-15 administration on other immunosuppressive pathways, such as Tim-3 (93-95), support evaluation of combinatorial treatment of hetIL-15 with appropriate checkpoint inhibitors in the context of ACT therapy.

In conclusion, the illustrative studies provided herein identified several benefits of hetIL-15 in combination with ACT for cancer immunotherapy. hetIL-15 administration improved the outcome of ACT in the absence of lymphodepletion providing a clear advantage over protocols using host lymphodepletion.

### REFERENCES CITED BY NUMBER

1. Klebanoff C A, Rosenberg S A, and Restifo N P. Prospects for gene-engineered T cell immunotherapy for solid cancers. Nat Med. 2016; 22(1):26-36.
2. Rosenberg S A, and Dudley M E. Adoptive cell therapy for the treatment of patients with metastatic melanoma. Current opinion in immunology, 2009:21(2):233-40.
3. Rosenberg S A, Restifo N P, Yang J C, Morgan R A, and Dudley M E. Adoptive cell transfer: a clinical path to effective cancer immunotherapy. Nature reviews Cancer, 2008:8(4):299-308.
4. Morgan R A, Dudley M E, Wunderlich J R, Hughes M S, Yang J C, Sherry R M, Royal R E, Topalian S L, Kammula U S, Restifo N P, et al. Cancer regression in patients after transfer of genetically engineered lymphocytes. Science, 2006; 314(5796): 126-9.
5. June C H, Riddell S R. and Schumacher T N. Adoptive cellular therapy: a race to the finish line. Science translational medicine. 2015; 7(280):280ps7.
6. Porter D L, Levine B L, Kalos M. Bagg A, and June C H. Chimeric antigen receptor-modified T cells in chronic lymphoid leukemia. N Engl J Med. 2011; 365(8):725-33.
7. Dudley M E, Wunderlich J R, Robbins P F, Yang J C, Hwu P, Schwartzentruber D J, Topalian S L, Sherry R, Restifo N P, Hubicki A M, et al. Cancer regression and autoimmunity in patients after clonal repopulation with antitumor lymphocytes. Science. 2002; 298(5594):850-4.
8. Wrzesinski C, Paulos C M, Kaiser A, Muranski P, Palmer D C, Gattinoni L, Yu Z. Rosenberg S A, and Restifo N P. Increased intensity lymphodepletion enhances tumor treatment efficacy of adoptively transferred tumor-specific T cells. J Immunother. 2010:33(1):1-7.
9. Dudley M E, Wunderlich J R, Yang J C, Sherry R M. Topalian S L, Restifo N P, Royal R E, Kammula U, White D E. Mavroukakis S A, et al. Adoptive cell transfer therapy following non-myeloablative but lymphodepleting chemotherapy for the treatment of patients with refractory metastatic melanoma. J Clin Oncol. 2005:23(10):2346-57.
10. Dudley M E, Yang J C, Sherry R. Hughes M S, Royal R, Kammula U, Robbins P F, Huang J, Citrin D E, Leitman S F, et al. Adoptive cell therapy for patients with metastatic melanoma: evaluation of intensive myeloablative chemoradiation preparative regimens. J Clin Oncol. 2008; 26(32):5233-9.
11. Gattinoni L, Finkelstein S E, Klebanoff C A, Antony P A, Palmer D C, Spiess P J, Hwang L N, Yu Z, Wrzesinski C, Heimann D M, et al. Removal of homeostatic cytokine sinks by lymphodepletion enhances the efficacy of adoptively transferred tumor-specific CD8+ T cells. J Exp Med. 2005; 202(7):907-12.
12. Klebanoff C A, Khong H T, Antony P A, Palmer D C, and Restifo N P, Sinks, suppressors and antigen presenters: how lymphodepletion enhances T cell-mediated tumor immunotherapy. Trends in immunology. 2005; 26(2):111-7.
13. Hakim F T, Cepeda R, Kaimei S, Mackall C L, McAtee N, Zujewski J, Cowan K, and Gress R E. Constraints on CD4 recovery postchemotherapy in adults: thymic insufficiency and apoptotic decline of expanded peripheral CD4 cells. Blood. 1997:90(9):3789-98.
14. Mackall C L, Fleisher T A, Brown M R, Andrich M P, Chen C C, Feuerstein I M, Horowitz M E, Magrath I T, Shad A T, Steinberg S M, et al. Age, thymopoiesis, and CD4+ T-lymphocyte regeneration after intensive chemotherapy. N Engl J Med. 1995; 332(3): 143-9.
15. Williams K M, Hakim F T, and Gress R E. T cell immune reconstitution following lymphodepletion. Seminars in immunology. 2007; 19(5):318-30.
16. Lorenzi A R. Clarke A M, Wooldridge T, Waldmann H, Hale G, Symmons D, Hazleman B L, and Isaacs J D. Morbidity and mortality in rheumatoid arthritis patients with prolonged therapy-induced lymphopenia: twelve-year outcomes. Arthritis and rheumatism. 2008; 58(2):370-5.
17. Storek J, Gooley T, Witherspoon R P, Sullivan K M, and Storb R. Infectious morbidity in long-term survivors of allogeneic marrow transplantation is associated with low CD4 T cell counts. American journal of hematology. 1997; 54(2): 131-8.
18. Liu S. Riley J. Rosenberg S, and Parkhurst M. Comparison of common gamma-chain cytokines, interleukin-2, interleukin-7, and interleukin-15 for the in vitro generation of human tumor-reactive T lymphocytes for adoptive cell transfer therapy. J Immunother. 2006:29(3):284-93.
19. Overwijk W W, and Schluns K S. Functions of gammaC cytokines in immune homeostasis: current and potential clinical applications. Clinical immunology. 2009; 132(2): 153-65.
20. Berard M. Brandt K, Bulfone-Paus S, and Tough D F. IL-15 promotes the survival of naive and memory phenotype CD8+ T cells. J Immunol. 2003; 170(10):5018-26.
21. Carson W E, Girl J G, Lindemann M J, Linett M L. Ahdieh M, Paxton R, Anderson D. Eisenmann J, Grabstein K, and Caligiuri M A. Interleukin (IL) 15 is a novel cytokine that activates human natural killer cells via components of the IL-2 receptor. J Exp Med. 1994:180(4): 1395-403.
22. Ma A, Koka R, and Burkett P. Diverse functions of IL-2, IL-15, and IL-7 in lymphoid homeostasis. Annu Rev Immunol. 2006; 24(657-79.
23. Zhang X, Sun S, Hwang I, Tough D F, and Sprent J. Potent and selective stimulation of memory-phenotype CD8+ T cells in vivo by IL-15. Immunity. 1998; 8(5):591-9.
24. Goldrath A W, Sivakumar P V, Glaccum M, Kennedy M K, Bevan M J, Benoist C. Mathis D, and Butz E A. Cytokine requirements for acute and Basal homeostatic proliferation of naive and memory CD8+ T cells. J Exp Med. 2002:195(12):1515-22.
25. Ramsey C, Rubinstein M P, Kim D M, Cho J H, Sprent J, and Surh C D. The lymphopenic environment of CD132 (common gamma-chain)-deficient hosts elicits rapid homeostatic proliferation of naive T cells via IL-15. J Immunol. 2008; 180(8):5320-6.
26. Klebanoff C A. Finkelstein S E, Surman D R, Lichtman M K, Gattinoni L, Theoret M R, Grewal N, Spiess P J, Antony P A, Palmer D C, et al. IL-15 enhances the in vivo antitumor activity of tumor-reactive CD8+ T cells. Proc Natl Acad Sci USA. 2004:101(7): 1969-74.
27. Berger C, Jensen M C, Lansdorp P M, Gough M, Elliott C, and Riddell S R. Adoptive transfer of effector CD8+ T cells derived from central memory cells establishes persistent T cell memory in primates. The Journal of clinical investigation. 2008; 118(1):294-305.
28. Oelert T, Papatriantafvllou M, Pougialis G, Hammerling G J, Arnold B, and Schuler T. Irradiation and IL-15 promote loss of CD8 T-cell tolerance in response to lymphopenia. Blood. 2010, 115(11):2196-202.
29. Teague R M, Sather B D, Sacks J A, Huang M Z, Dossett M L, Morimoto J, Tan X,
Sutton S E, Cooke M P, Ohlen C, et al. Interleukin-15 rescues tolerant CD8+ T cells for use in adoptive immunotherapy of established tumors. Nat Med. 2006; 12(3):335-41.
30. Boussiotis V A, Barber D L, Nakarai T, Freeman G J, Gribben J G, Bernstein G M, D'Andrea A D, Ritz J. and Nadler L M. Prevention of T cell anergy by signaling through the gamma c chain of the IL-2 receptor. Science. 1994:266(5187):1039-42.
31. Bergamaschi C, Bear J, Rosati M, Beach R K, Alicca C, Sowder R, Chertova E, Rosenberg S A, Felber B K, and Pavlakis G N. Circulating IL-15 exists as heterodimeric complex with soluble IL-15Ralpha in human and mouse serum. Blood. 2012; 120(1):e1-8.
32. Bergamaschi C, Jalah R, Kulkarni V, Rosati M, Zhang G M, Alicca C, Zolotukhin A S, Felber B K. and Pavlakis G N. Secretion and biological activity of short signal peptide IL-15 is chaperoned by IL-15 receptor alpha in vivo. J Immunol. 2009; 183(5):3064-72.
33. Bergamaschi C, Rosati M, Jalah R. Valentin A, Kulkami V. Alicea C. Zhang G M, Patel V, Felber B K, and Pavlakis G N. Intracellular Interaction of Interleukin-15 with Its Receptor {alpha} during Production Leads to Mutual Stabilization and Increased Bioactivity. J Biol Chem. 2008:283(7):4189-99.
34. Mortier E, Woo T, Advincula R, Gozalo S, and Ma A. IL-15Ralpha chaperones IL-15 to stable dendritic cell membrane complexes that activate N K cells via trans presentation. J Exp Med. 2008; 205(5):1213-25.
35. Dubois S, Mariner J. Waldmann T A, and Tagaya Y. IL-15Ralpha recycles and presents IL-15 In trans to neighboring cells. Immunity. 2002; 17(5):537-47.
36. Rubinstein M P, Kovar M, Purton J F, Cho J H, Boyman O, Surh C D, and Sprent J. Converting IL-15 to a superagonist by binding to soluble IL-15R{alpha}. Proc Natl Acad Sci USA. 2006:103(24):9166-71.
37. Chertova E, Bergamaschi C, Chertov O, Sowder R I Bear J, Roser J D, Beach R K, Lifson J D, Felber B K, and Pavlakis G N. Characterization and favorable in vivo properties of heterodimeric soluble IL-15.IL-15Ralpha cytokine compared to IL-15 monomer. J Biol Chem. 2013; 288(25): 18093-103.
38. Overwijk W W, Theoret M R, Finkelstein S E. Surman D R, de Jong L A, Vyth-Dreese F A, Dellemijn T A, Antony P A, Spiess P J, Palmer D C, et al. Tumor regression and autoimmunity after reversal of a functionally tolerant state of self-reactive CD8+ T cells. J Exp Med. 2003; 198(4):569-80.
39. Fehniger T A, Cai S F, Cao X, Bredemeyer A J, Presti R M, French A R, and Ley T J. Acquisition of murine N K cell cytotoxicity requires the translation of a pre-existing pool of granzyme B and perforin mRNAs. Immunity. 2007; 26(6):798-811.
40. Ye W. Young J D, and Liu C C. Interleukin-15 induces the expression of mRNAs of cytolytic mediators and augments cytotoxic activities in primary murine lymphocytes. Cellular immunology. 1996:174(1):54-62.
41. Pardoll D M. The blockade of immune checkpoints in cancer immunotherapy. Nature reviews Cancer. 2012:12(4):252-64.
42. Dong H, Strome S E, Salomao D R, Tamura H, Hirano F, Flies D B, Roche P C, Lu J, Zhu G, Tamada K, et al. Tumor-associated B7-H1 promotes T-cell apoptosis: a potential mechanism of immune evasion. Nat Med. 2002:8(8):793-800.
43. Ahmadzadeh M, Johnson L A, Heemskerk B. Wunderlich J R, Dudley M E, White D E, and Rosenberg S A. Tumor antigen-specific CD8 T cells infiltrating the tumor express high levels of PD-1 and are functionally impaired. Blood. 2009; 114(8):1537-44.
44. Baitsch L, Baumgaertner P, Devevre E, Raghav S K, Legat A, Barba L, Wieckowski S, Bouzourene H, Deplancke B. Romero P. et al. Exhaustion of tumor-specific CD8(+) T cells in metastases from melanoma patients. The Journal of clinical investigation. 2011; 121(6):2350-60.
45. Dubois S, Patel H J, Zhang M, Waldmann T A, and Muller J R. Preassociation of IL-15 with IL-15R alpha-IgG1-Fc enhances its activity on proliferation of N K and CD8+/CD44high T cells and its antitumor action. J Immunol. 2008:180(4):2099-106.
46. Epardaud M, Elpek K G, Rubinstein M P, Yonekura A R, Bellemare-Pelletier A. Bronson R, Hamerman J A, Goldrath A W, and Turley S J. Interleukin-15/interleukin-15R alpha complexes promote destruction of established tumors by reviving tumor-resident CD8+ T cells. Cancer research. 2008; 68(8):2972-83.
47. Kobayashi H. Dubois S. Sato N, Sabzevari H, Sakai Y, Waldmann T A, and Tagaya Y. Role of trans-cellular IL-15 presentation in the activation of N K cell-mediated killing, which leads to enhanced tumor immunosurveillance. Blood. 2005; 105(2):721-7.
48. Stoklasek T A, Schluns K S, and Lefrancois L. Combined IL-15/IL-15Ralpha immunotherapy maximizes IL-15 activity in vivo. J Immunol. 2006; 177(9):6072-80.
49. Wu J. IL-15 Agonists: The Cancer Cure Cytokine. Journal of molecular and genetic medicine: an international journal of biomedical research. 2013; 7(85.
50. Hong E, Usiskin I M, Bergamaschi C. Hanlon D J, Edelson R L, Justesen S. Pavlakis G N, Flavell R A, and Fahmy T M. Configuration-dependent presentation of multivalent IL-15:IL-15Ralpha enhances the antigen-specific T cell response and anti-tumor immunity. J Biol Chem. 2015.
51. Klebanoff C A, Gattinoni L, Palmer D C, Muranski P, Ji Y, Hinrichs C S, Borman Z A, Kerkar S P, Scott C D, Finkelstein S E, et al. Determinants of successful CD8+ T-cell adoptive immunotherapy for large established tumors in mice. Clinical cancer research: an official journal of the American Association for Cancer Research. 2011; 17(16):5343-52.
52. Tseng J, Citrin D E, Waldman M, White D E, Rosenberg S A, and Yang J C. Thrombotic microangiopathy in metastatic melanoma patients treated with adoptive cell therapy and total body irradiation. Cancer. 2014; 120(9): 1426-32.
53. Conlon K C, Lugli E, Welles H C. Rosenberg S A, Fojo A T, Morris J C, Fleisher T A, Dubois S P. Perera L P, Stewart D M, et al. Redistribution, Hyperproliferation, Activation of Natural Killer Cells and CD8 T Cells, and Cytokine Production During First-in-Human Clinical Trial of Recombinant Human Interleukin-15 in Patients With Cancer. J Clin Oncol. 2014.
54. Waldmann T A, Lugli E. Roederer M, Perera L P, Smedley J V, Macallister R P, Goldman C K, Bryant B R. Decker J M, Fleisher T A, et al. Safety (toxicity), pharmacokinetics, immunogenicity, and impact on elements of the normal immune system of recombinant human IL-15 in rhesus macaques. Blood. 2011:117(18):4787-95.
55. de Witte M A. Jorritsma A, Kaiser A, van den Boom M D, Dokter M, Bendle G M. Haanen J B, and Schumacher T N. Requirements for effective antitumor responses of TCR transduced T cells. J Immunol. 2008; 181(7):5128-36.
56. Kline J, Zhang L, Battaglia L, Cohen K S, and Gajewski T F. Cellular and molecular requirements for rejection of B16 melanoma in the setting of regulatory T cell depletion and homeostatic proliferation. J Immunol. 2012:188(6):2630-42.
57. Pule M A, Savoldo B. Myers G D, Rossig C. Russell H V, Dotti G, Huls M H, Liu E, Gee A P, Mei Z, et al. Virus-specific T cells engineered to coexpress tumor-specific receptors; persistence and antitumor activity in individuals with neuroblastoma. Nat Med. 2008:14(11):1264-70.
58. Robbins P F, Dudley M E, Wunderlich J. El-Gamil M, Li Y F, Zhou J, Huang J, Powell D J, Jr., and Rosenberg S A. Cutting edge: persistence of transferred lymphocyte clonotypes correlates with cancer regression in patients receiving cell transfer therapy. J Immunol. 2004:173(12):7125-30.
59. Zhou J, Shen X, Huang J, Hodes R J, Rosenberg S A, and Robbins P F. Telomere length of transferred lymphocytes correlates with in vivo persistence and tumor regression in melanoma patients receiving cell transfer therapy. J Immunol. 2005:175(10):7046-52.
60. Yee C, Thompson J A, Byrd D, Riddell S R, Roche P. Celis E, and Greenberg P D. Adoptive T cell therapy using antigen-specific CD8+ T cell clones for the treatment of patients with metastatic melanoma: in vivo persistence, migration, and antitumor effect of transferred T cells. Proc Natl Acad Sci USA. 2002:99(25):16168-73.
61. Spranger S. Koblish H K, Horton B, Scherle P A, Newton R, and Gajewski T F. Mechanism of tumor rejection with doublets of CTLA-4, PD-1/PD-L1, or IDO blockade involves restored IL-2 production and proliferation of CD8(+) T cells directly within the tumor microenvironment. Journal for immunotherapy of cancer. 2014; 2(3.
62. Gattinoni L, Klebanoff C A, Palmer D C, Wrzesinski C. Kerstann K. Yu Z, Finkelstein S E, Theoret M R, Rosenberg S A, and Restifo N P. Acquisition of full effector function in vitro paradoxically impairs the in vivo antitumor efficacy of adoptively transferred CD8+ T cells. The Journal of clinical investigation. 2005; 115(6): 1616-26.
63. Gattinoni L, Lugli E, Ji Y, Pos Z, Paulos C M, Quigley M F, Almeida J R, Gostick E, Yu Z, Carpenito C. et al. A human memory T cell subset with stem cell-like properties. Nat Med. 2011:17(10):1290-7.
64. Hinrichs C S, Borman Z A, Cassard L. Gattinoni L, Spolski R, Yu Z, Sanchez-Perez L, Muranski P, Kern S J, Logun C. et al. Adoptively transferred effector cells derived from naive rather than central memory CD8+ T cells mediate superior antitumor immunity. Proc Natl Acad Sci USA. 2009; 106(41):17469-74.
65. Klebanoff C A, Gattinoni L, Torabi-Parizi P, Kerstann K, Cardones A R, Finkelstein S E, Palmer D C, Antony P A, Hwang S T, Rosenberg S A, et al. Central memory self/tumor-reactive CD8+ T cells confer superior antitumor immunity compared with effector memory T cells. Proc Natl Acad Sci USA. 2005:102(27):9571-6.
66. Klebanoff C A, Scott C D, Leonardi A J, Yamamoto T N, Cruz A C, Ouyang C, Ramaswamy M, Roychoudhuri R, Ji Y, Eil R L, et al. Memory T cell-driven differentiation of naive cells impairs adoptive immunotherapy. The Journal of clinical investigation. 2016; 126(1):318-34.
67. Cui Y, Zhang H, Meadors J, Poon R, Guimond M. and Mackall C L. Harnessing the physiology of lymphopenia to support adoptive immunotherapy in lymphoreplete hosts. Blood. 2009; 114(18):3831-40.
68. Rech A J, Mick R Martin S. Recio A, Aqui N A, Powell D J, Jr., Colligon T A, Trosko J A, Leinbach L I, Pletcher C H, et al. CD25 blockade depletes and selectively reprograms regulatory T cells in concert with immunotherapy in cancer patients. Science translational medicine. 2012; 4(134): 134ra62.
69. Ji Y. Wrzesinski C, Yu Z, Hu J, Gautam S, Hawk N V, Telford W G, Palmer D C, Franco Z, Sukumar M, et al. miR-155 augments CD8+ T-cell antitumor activity in lymphoreplete hosts by enhancing responsiveness to homeostatic gammac cytokines. Proc Natl Acad Sci USA. 2015:112(2):476-81.
70. Kerkar S P, Muranski P, Kaiser A, Boni A, Sanchez-Perez L, Yu Z, Palmer D C, Reger R N, Borman Z A, Zhang L, et al. Tumor-specific CD8+ T cells expressing interleukin-eradicate established cancers in lymphodepleted hosts. Cancer research. 2010; 70(17):6725-34.
71. Cho H I, Reyes-Vargas E. Delgado J C, and Celis E. A potent vaccination strategy that circumvents lymphodepletion for effective antitumor adoptive T-cell therapy. Cancer research. 2012; 72(8): 1986-95.
72. Su E W, Moore C J, Suriano S, Johnson C B, Songalia N. Patterson A, Neitzke D J, Andrijauskaite K, Garrett-Mayer E. Mehrotra S, et al. IL-2Ralpha mediates temporal regulation of IL-2 signaling and enhances immunotherapy. Science translational medicine. 2015:7(311):311ra170.
73. Church S E, Jensen S M, Antony P A. Restifo N P. and Fox B A. Tumor-specific CD4+ T cells maintain effector and memory tumor-specific CD8+ T cells. European journal of immunology. 2014; 44(1):69-79.
74. Hu H M, Winter H. Urba W J. and Fox B A. Divergent roles for CD4+ T cells in the priming and effector/memory phases of adoptive immunotherapy. J Immunol. 2000; 165(8):4246-53.
75. Malandro N, Budhu S, Kuhn N F, Liu C, Murphy J T, Cortez C, Zhong H. Yang X, Rizzuto G, Altan-Bonnet G, et al. Clonal Abundance of Tumor-Specific CD4(+) T Cells Potentiates Efficacy and Alters Susceptibility to Exhaustion. Immunity. 2016; 44(1): 179-93.
76. Hirschhom-Cymerman D, Budhu S. Kitano S, Liu C. Zhao F, Zhong H. Lesokhin A M. Avogadri-Connors F, Yuan J, Li Y, et al. Induction of tumoricidal function in CD4+ T cells is associated with concomitant memory and terminally differentiated phenotype. J Exp Med. 2012; 209(11):2113-26.
77. Perales M A, and Wolchok J D. CD4 help and tumor immunity: beyond the activation of cytotoxic T lymphocytes. Annals of surgical oncology. 2004; 11(10):881-2.
78. Bergamaschi C, Kulkami V, Rosati M, Alicea C, Jalah R, Chen S, Bear J. Sardesai N Y. Valentin A, Felber B K. et al. Intramuscular delivery of heterodimeric IL-15 DNA in macaques produces systemic levels of bioactive cytokine inducing proliferation of N K and T cells. Gene therapy. 2015:22(1):76-86.
79. Picker L J, Reed-Indcrbitzin E F, Hagen S I, Edgar J B, Hansen S G, Legasse A, Planer S, Piatak M, Jr., Lifson J D, Maino V C, et al. IL-15 induces CD4 effector memory T cell production and tissue emigration in nonhuman primates. The Journal of clinical investigation. 2006; 116(6):1514-24.
80. Gajewski T F, Woo S R. Zha Y, Spaapen R, Zheng Y. Corrales L. and Spranger S. Cancer immunotherapy strategies based on overcoming barriers within the tumor microenvironment. Current opinion in immunology. 2013; 25(2):268-76.
81. Spranger S, and Gajewski T. Rational combinations of immunotherapeutics that target discrete pathways. Journal for immunotherapy of cancer. 2013; 1(16.
82. Spranger S, Spaapen R M, Zha Y, Williams J, Meng Y, Ha T T, and Gajewski T F. Up-regulation of PD-L1. IDO, and T(regs) in the melanoma tumor microenvironment is driven by CD8(+) T cells. Science translational medicine. 2013; 5(200):200ral 16.
83. Palmer D C, Balasubramaniam S, Hanada K. Wrzesinski C, Yu Z, Farid S, Theoret M R, Hwang L N, Klebanoff C A, Gattinoni L. et al. Vaccine-stimulated, adoptively transferred CD8+ T cells traffic indiscriminately and ubiquitously while mediating specific tumor destruction. J Immunol. 2004; 173(12):7209-16.
84. Appay V. Jandus C, Voelter V, Reynard S. Coupland S E, Rimoldi D, Lienard D, Guillaume P. Krieg A M, Cerottini J C, et al. New generation vaccine induces effective melanoma-specific CD8+ T cells in the circulation but not in the tumor site. J Immunol. 2006; 177(3):1670-8.
85. Hailemichael Y, Dai Z, Jaffarzad N, Ye Y, Medina M A, Huang X F, Dorta-Estremera S M, Greeley N R, Nitti G, Peng W, et al. Persistent antigen at vaccination sites induces tumor-specific CD8(+) T cell sequestration, dysfunction and deletion. Nat Med. 2013:19(4):465-72.
86. Melchionda F, Fry T J, Milliron M J, McKirdy M A, Tagaya Y, and Mackall C L. Adjuvant IL-7 or IL-15 overcomes immunodominance and improves survival of the CD8+ memory cell pool. The Journal of clinical investigation. 2005; 115(5):1177-87.
87. Hodi F S, O'Day S J, McDermott D F, Weber R W, Sosman J A, Haanen J B, Gonzalez R, Robert C, Schadendorf D, Hassel J C, et al. Improved survival with ipilimumab in patients with metastatic melanoma. N Engl J Med. 2010; 363(8):711-23.
88. Jensen S M, Maston L D, Gough M J, Ruby C E, Redmond W L. Crittenden M, Li Y. Puri S, Poehlein C H Morris N. et al. Signaling through OX40 enhances antitumor immunity. Seminars in oncology. 2010:37(5):524-32.
89. Brahmer J R, Tykodi S S, Chow L Q. Hwu W J. Topalian S L, Hwu P, Drake C G. Camacho L H, Kauh J, Odunsi K, et al. Safety and activity of anti-PD-L1 antibody in patients with advanced cancer. N Engl J Med. 2012:366(26):2455-65.
90. Topalian S L, Hodi F S, Brahmer J R, Gettinger S N, Smith D C, McDermott D F, Powderly J D, Carvajal R D, Sosman J A, Atkins M B, et al. Safety, activity, and immune correlates of anti-PD-1 antibody in cancer. N Engl J Med. 2012; 366(26):2443-54.
91. Kao C, Oestreich K J, Paley M A. Crawford A, Angelosanto J M, Ali M A, Intlekofer A M, Boss J M, Reiner S L, Weinmann A S, et al. Transcription factor T-bet represses expression of the inhibitory receptor PD-1 and sustains virus-specific CD8+ T cell responses during chronic infection. Nat Immunol. 2011; 12(7):663-71.
92. Intlekofer A M, Takemoto N, Wherry E J, Longworth S A, Northrup J T, Palanivel V R. Mullen A C, Gasink C R, Kaech S M. Miller J D, et al. Effector and memory CD8+ T cell fate coupled by T-bet and eomesodermin. Nat Immunol. 2005; 6(12):1236-44.
93. Heon E K, Wulan H, Macdonald L P, Malek A O, Braunstein G H, Eaves C G, Schattner M D, Allen P M, Alexander M O, Hawkins C A, et al. IL-15 induces strong but short-lived tumor-infiltrating CD8 T cell responses through the regulation of Tim-3 in breast cancer. Biochemical and biophysical research communications. 2015; 464(1):360-6.
94. Anderson A C. Tim-3, a negative regulator of anti-tumor immunity. Current opinion in immunology. 2012:24(2):213-6.
95. Sakuishi K, Apetoh L, Sullivan J M, Blazar B R, Kuchroo V K, and Anderson A C. Targeting Tim-3 and PD-1 pathways to reverse T cell exhaustion and restore anti-tumor immunity. J Exp Med. 2010; 207(10):2187-94.
96. Thaysen-Andersen M. Chertova E, Bergamaschi C, Moh E S, Chertov O, Roser J, Sowder R, Bear J, Lifson J, Packer N H, et al. Recombinant human heterodimeric IL-15 complex displays extensive and reproducible N- and O-linked glycosylation. Glycoconjugate Journal. 2015.
97. Feng Z, Jensen S M, Messenheimer D J, Farhad M. Neuberger M, Bifulco C B, and Fox B A. Multispectral Imaging of T and B Cells in Murine Spleen and Tumor. J Immunol. 2016.
98. Sato E. et al., Intraepithelial CD8+ tumor-infiltrating lymphocytes and a high CD8+/regulatory T cell ratio are associated with favorable prognosis in ovarian cancer. Proc. Natl. Acad. Sci. 102:18538-43, 2005.

It is understood that the examples and embodiments described herein are for illustrative purposes only and that various modifications or changes in light thereof will be suggested to persons skilled in the art and are to be included within the spirit and purview of this application and scope of the appended claims. All publications, patents, accession numbers, and patent applications cited herein are hereby incorporated by reference in their entirety for all purposes.

1. A method of increasing adoptive cell therapy efficacy in a subject that does not undergo a lymphodepletion procedure, the method comprising:
administering a heterodimeric IL-15/IL-15 receptor alpha complex (hetIL-15) to the subject;
administering adoptive cell transfer (ACT) cells to the subject, wherein hetIL-15 is administered at a frequency and in an amount that increases the number of lymphocytes present in the tumor.

2. The method of claim 1, wherein hetIL-15 is administered for at least 10 days.

3. The method of claim 1, wherein het IL-15 is administered every day.

4. The method of claim 2, wherein het IL-15 is administered every day.

5. The method of claim 1, wherein hetIL-15 is administered at two-day intervals or at three-day intervals.

6. The method of claim 2, wherein hetIL-15 is administered at two-day intervals or at three-day intervals.

7. The method of claim 1, wherein the ACT cell comprise CD8+ lymphocytes.

8. The method of claim 1, wherein the ACT cell comprise Natural Killer cells.

9. The method of claim 1, wherein the ACT cells are genetically modified to enhance anti-tumor effects.

10. The method of claim 1, wherein hetIL-15 is administered subcutaneously.

11. The method of claim 1, wherein hetIL-15 is administered intravenously.

12. The method of claim 1, wherein the IL-15 receptor alpha present in hetIL-15 comprises soluble IL-15 receptor alpha form that is not fused to an Fc region.

13. The method of claim 1, wherein the IL-15 receptor alpha present in het IL-15 comprises an IL-15 receptor alpha-Fc fusion polypeptide.

14. The method of claim 1, wherein the subject is a human.


SEQUENCE LISTING (13 sequences)

SEQ ID NO: 1 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - DNA, 489 bp, Homo sapiens
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 3 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 4 - protein, 200 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 5 - DNA, 804 bp, Homo sapiens
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatga

SEQ ID NO: 6 - DNA, 600 bp, Homo sapiens
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggc

SEQ ID NO: 7 - protein, 205 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 8 - protein, 8 aa, Artificial Sequence
  Synthetic construct
PQGHSDTT

SEQ ID NO: 9 - protein, 7 aa, Artificial Sequence
  Synthetic construct
PQGHSDT

SEQ ID NO: 10 - protein, 6 aa, Artificial Sequence
  Synthetic construct
PQGHSD

SEQ ID NO: 11 - protein, 5 aa, Artificial Sequence
  Synthetic construct
PQGHS

SEQ ID NO: 12 - protein, 4 aa, Artificial Sequence
  Synthetic construct
PQGH

SEQ ID NO: 13 - DNA, 615 bp, Homo sapiens
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccact